(19) United States
(12) Patent Application Publication
(10) Pub. No.: US 2025/0249089 A1
(43) Pub. Date: Aug. 7, 2025

(54) RECOMBINANT PLASMID, IMMUNOGENIC COMPOSITION, KIT AND USES THEREOF

(71) Applicant: FUNDAÇÃO OSWALDO CRUZ (FIOCRUZ), Rio de Janeiro (BR)

(72) Inventors: RICARDO TOSTES GAZZINELLI, BELO HORIZONTE (BR); NATÁLIA SATCHIKO HOJO DE SOUZA, BELO HORIZONTE (BR); PATRICK ORESTES DE AZEVEDO, BELO HORIZONTE (BR); EMILIANO ROSA OLIVEIRA, BELO HORIZONTE (BR); SANTUZA MARIA RIBEIRO TEXEIRA, BELO HORIZONTE (BR); ANA PAULA SALLES MOURA FERNANDES, BELO HORIZONTE (BR); DANIEL HENRIQUE DORO PEREIRA, BELO HORIZONTE (BR)

(73) Assignee: FUNDAÇÃO OSWALDO CRUZ (FIOCRUZ), Rio de Janeiro (BR)

(21) Appl. No.: 19/027,243

(22) Filed: Jan. 17, 2025

(30) Foreign Application Priority Data
Feb. 7, 2024 (BR) 1020240025377

(51) Int. Cl. A61K 39/215; A61K 39/00; A61K 39/145; A61P 31/14; C12N 7/00; C12N 15/85

(52) U.S. Cl. CPC A61K 39/215; A61K 39/145; A61P 31/14; C12N 7/00; C12N 15/85; A61K 2039/53; A61K 2039/545; A61K 2039/575; A61K 2039/70; C12N 2760/16134; C12N 2770/20034

(57) ABSTRACT

Recombinant plasmids and immunogenic DNA compositions including a recombinant plasmid which carries the gene sequence encoding the Spike protein or the gene sequence encoding the NP protein, with the capacity to prevent high viral loads and moderate and severe clinical forms of the disease by stimulating the immune system, both against the ancestral strain of SARS-CoV-2 and influenza strains against different variants of concern that are currently circulating are disclosed. A bivalent recombinant plasmid and a immunogenic composition including the bivalent recombinant plasmid which carries the gene sequence encoding the NP and Spike protein, with the capacity to prevent high viral loads and moderate and severe clinical forms of the disease by stimulating the immune system, both against the influenza strain and SARS-COV-2 strain against different variants of concern that are currently circulating are disclosed.

### CROSS-REFERENCE TO RELATED APPLICATION

This application is based on and claims the benefit of priority 1 from Brazilian patent application Ser. No. 1020240025377, filed on Feb. 7, 2024, the entirety of which is incorporated by reference herein.

### REFERENCE TO SEQUENCE LISTING

The instant application contains a sequence listing which has been submitted electronically in XML format and is hereby incorporated by reference in its entirety. Said XML copy, created on Dec. 13, 2024, is named “PE003546—NWE SEQUENCE LISTING—. xml” and is 85, 647 bytes in size.

### FIELD OF THE INVENTION

The present invention is found in the field of biotechnology. More precisely the present invention refers to the development of a vaccine platform that consists of two plasmids pCTV1 and pCTV2, produced from the modification of a commercial plasmid (pCDNA 3.1) so that only the genes which are essential for the expression of proteins in mammalian cells are maintained. The present platform can be used in the development of vaccines for different diseases, as an example, COVID-19, seasonal influenza, dengue, malaria and leishmaniasis. In this way, the present invention also encompasses recombinant plasmids and immunogenic compositions against COVID-19 and Influenza using the platform.

### BACKGROUND OF THE INVENTION

The use of plasmids (DAN) as vectors for the expression of genes of interest has more than thirty years of study. In 1990, Wolff et al. performed a study in which they injected expression plasmid vectors comprising genes for chloramphenicol acetyltransferase, luciferase and β-galactosidase in mouse skeletal muscle and observed the expression of proteins in the muscle cells of these animals. This approach suggested that it was possible to obtain intracellular expression of genes encoding antigens and would be an alternative for vaccine development (WOLFF et al., 1990). These observations triggered a series of investigations using DNA plasmids, aiming to induce immune responses with the direct injection of DNA encoding antigenic proteins in preclinical animal models for viral, bacterial, and parasitic diseases. In 1993, Ulmer et al. using plasmid DNA which carries the Influenza A virus nucleoprotein gene demonstrated in vivo expression of the protein as well as protection of animals that were challenged with the Influenza A virus. These findings demonstrated the feasibility of using plasmid DNA as a vaccine platform.

This type of vaccine platform consists primarily of a bacterial DNA plasmid linked to a gene of interest, a viral promoter, and a transcriptional termination sequence. After the stage of linking the gene of interest, the plasmid is transformed to insert it into the bacteria, the transformation product is expanded and, subsequently, the purification of the plasmid is performed. After confirming the correct construction of the plasmid, it is injected into the host that will be tested, thus, this plasmid is translocated to the nucleus of the host cell and the viral promoter is activated, resulting in the transcription of the gene of interest and production of the protein, which induces the humoral and cellular immune response.

The use of DNA vaccines allows an immediate response in the case of emerging endemic/pandemic pathogens with a high transmission rate, as well as in the case of the emergence of new variants of SARS-COV-2, since the process of replacing protein-coding genes is relatively simple and rapid. There are currently some DNA vaccines approved for the treatment of diseases of veterinary interest such as West Nile virus in horses, infectious hematopoietic necrosis virus in salmon and canine melanoma, as well as several preclinical studies using this vaccine platform. DNA-based vaccines for COVID-19, still in development, have shown promise and consistent results in stimulating humoral and cellular immune responses. DNA vaccines are likely to require booster doses as well, but they have other advantages, such as low cost, high stability at room temperature and easy distribution. Furthermore, this platform allows an immediate response in the case of emerging endemic/pandemic diseases of pathogens with a high transmission rate.

Despite a relatively large global vaccination program against COVID-19, unequal access to vaccines between high- and low-income countries is a constant threat for the emergence of variants of concern of SARS-COV-2 (VOCs). With the emergence of Omicron, we are now facing the fourth wave of the pandemic. Difficulties in eliminating SARS-COV-2 and the emergence of VOCs capable of immune evasion have required booster doses. However, even after the fourth dose, current COVID-19 vaccines induce low levels of nAbs that fail to control viral replication and prevent mild to moderate disease caused by the Omicron variant. This suggests that COVID-19 may become an endemic disease that should be included in the periodic vaccination schedule in several countries.

The race to find vaccines has resulted in the development of different vaccine platforms, with varying levels of efficacy and efficiency. The third generation of vaccines employs the strategy of transferring nucleic acids encoding antigens into the host, which are then expressed by the host cells. mRNA-based vaccines against COVID-19 have shown high efficacy and have been widely used worldwide. Furthermore, vaccines based on non-replicating viral vectors such as recombinant human adenovirus type 26 (Ad26) and modified chimpanzee adenovirus (ChadOx1) have proven effective and have been distributed in different countries.

Vaccines approved for use against COVID-19 are based on neutralizing antibodies (nAbs), however, the emergence of new variants and subvariants of SARS-COV-2 capable of evading the action of nAbs are contributing to the maintenance of the disease. Furthermore, the decline in antibody titers is observed in mRNA vaccines after approximately 6 months, therefore requiring booster doses. Recent studies have shown that the memory T cell responses of infected and/or vaccinated individuals are more conserved and largely preserved for the Omicron Spike and non-Spike proteins. Furthermore, booster vaccination increased T cell reactivity to Omicron Spike. Thus, it is possible that protection against severe disease among vaccinated individuals is mediated by cellular immune responses.

In this context, a DNA-based platform that induces a strong protective immune response mediated by the T cell response is an important strategy to rapidly adapt vaccines against SARS-COV-2 and its VOCs.

There are patents/patent applications that describe the development of vaccine platforms for the production of DNA vaccines, as well as vaccines against SARS-COV-2 that use DNA sequence encoding the Spike protein.

The international patent application WO 2022/071513, filed on Sep. 30, 2021, in the name of OSAKA UNIVERSITY; TAKARA BIO INC. and ANGES, INC., and entitled: “IMPROVED DNA VACCINE FOR SARS-COV-2” describes a DNA that encodes the coronavirus spike protein (SARS COV-2) or a fragment thereof; and has been optimized to partially or fully display a codon included in a DNA sequence.

The international patent application WO 2021/214703, filed on Apr. 22, 2021, in the name of ZYDUS LIFESCIENCES LIMITED, and entitled: “A VACCINE AGAINST SARS-COV-2 AND PREPARATION THEREOF” reveals a DNA construct comprising the S gene or the S1 gene region of the Spike protein-S 2019-nCOV. The vector may further comprise a gene encoding the IgE signal peptide or a gene encoding the t-PA signal peptide. The DNA construct, according to the international patent application is further used in the preparation of immunogenic composition or a vaccine to treat or prevent the coronavirus or its related diseases.

The international patent application WO 2022/081707, filed on Oct. 13, 2021, in the name of THE GOVERNMENT OF THE UNITED STATES, AS REPRESENTED BY THE SECRETARY OF THE ARMY, and entitled: “SARS-COV-2 DNA VACCINE AND METHOD OF ADMINISTERING THEREOF” provides a severe acute respiratory syndrome coronavirus 2 (SARS-COV-2) spike-based DNA vaccine capable of inducing an immune response to SARS-COV-2 in humans following administration.

The international patent application WO 2005/118813, filed on Jun. 3, 2005, in the name of INSTITUT PASTEUR and HKU-PASTEUR RESEARCH CENTRE LIMITED, and entitled: “NUCLEIC ACIDS, METHODS POLYPEPTIDES, OF EXPRESSION, AND IMMUNOGENIC COMPOSITIONS ASSOCIATED WITH SARS CORONA VIRUS SPIKE PROTEIN” reveal acid molecules, polypeptides, nucleic immunogenic compositions, vaccines and methods of manufacturing and use of nucleotides and polypeptides encoded associated with the Spike protein of SARS Coronavirus (SARS COV).

The international patent application WO 2022/011092, filed on Jul. 8, 2021, in the name of THE TRUSTEES OF THE UNIVERSITY OF PENNSYLVANIA, and entitled: “NUCLEOSIDE-MODIFIED RNA FOR INDUCING AN IMMUNE RESPONSE AGAINST SARS-COV-2” refers to compositions and methods for inducing an adaptive immune response against severe acute respiratory syndrome coronavirus 2 (SARS-COV-2) in a subject. In certain embodiments, the international patent application provides a composition comprising a nucleic acid molecule modified by nucleoside encoding a SARS-COV-2 antigen, adjuvant, or a combination thereof.

The US patent application 2021/338803, filed on May 14, 2020, in the name of SICHUAN CHENGYU BIOLOGICAL PRODUCTS INC., and entitled: “DUAL-MOLECULAR DNA VACCINE COMPOSED OF A VIRAL ANTIGEN AND AN IMMUNE COSTIMULATOR” describes a dual-molecular DNA vaccine system comprising a viral antigen and an immunization coactivator. Such vector is a DNA plasmid; the viral antigen is any immunogenic viral molecule (antigen); the immune activator is a kind of T cell co-stimulator. The vaccine disclosed in the present US patent application are two gene fragments that express the T cell co-stimulator and the viral antigen molecule, which are constructed into a DNA plasmid and will be co-expressed in a cell at the same time to activate the systemic immune response through both signaling. The dual-molecular DNA vaccine is also a novel vaccine technology platform used for the development of multiple vaccines to prevent various infectious diseases. Particularly, the dual-molecular DNA vaccine with the SARS-COV-2 S antigen and the T cells co-stimulator can be constructed through this platform to prevent and control the global COVID-19 pandemic.

The international patent application WO 2021/236854, filed on May 15, 2021, in the name of GRITSTONE BIO, INC., and entitled: “SARS-COV-2 VACCINES” describes vaccine compositions that include cassettes encoding SARS-COV-2 MHC epitopes and for full-length SARS-COV-2 proteins. Also disclosed are nucleotides, cells and methods associated with the compositions, including their use as vaccines.

The article published by James A. Williams et al. in Biotechnology Advances vol. 27, 4 (2009): 353-70 and entitled “PLASMID DNA VACCINE VECTOR DESIGN: IMPACT ON EFFICACY, SAFETY AND UPSTREAM PRODUCTION” describes the advantages of developing a reduced plasmid for vaccine use, including facilitating purification, removing antigenic peptides and making it a more potent vector. The article reports that such a reduction is not simple and may generate new problems.

The US patent application US 2021/338803 filed on May 14, 2020, and published on Nov. 4, 2021, in the name of SICHUAN CHENGYU BIOLOGICAL PRODUCTS INC., and entitled “DUAL-MOLECULAR DNA VACCINE COMPOSED OF A VIRAL ANTIGEN AND AN IMMUNE COSTIMULATOR” discloses a double DNA molecular vaccine system composed of viral antigen and immunization coactivator. Such vector is a DNA plasmid; the viral antigen is any immunogenic viral molecule (antigen); the immune activator is a kind of T cell co-stimulator. The difference between the vaccine revealed by the US patent application US 2021/338803 and the vaccine disclosed in the present invention is that two gene fragments expressing the T cell co-stimulator and the viral antigen molecule are constructed in a DNA plasmid and will be co-expressed in a cell at the same time to activate the systemic immune response through the two signaling.

In the state of the art, most of the prior art documents point out that the efficiency of vaccines is also associated with optimized codons, which are also comprised in the present application. Thus, the present application reveals that the use of plasmids reduced in vaccine platforms may also improve the immunogenic properties of the vaccine for SARS-COV-2 and/or Influenza. However, the process of constructing and using reduced plasmids is not trivial, and can generate results that are not satisfactory, as pointed out in the article published by James A. Williams. In this sense, in order to overcome the technical barriers in using reduced plasmids, the present application performed in vivo testing for SARS-COV-2 variants, which presented promising results for a potential vaccine candidate. The modification of the commercial plasmid to a reduced plasmid generated an unexpected effect that impacted improvements in the immunogenic properties of the vaccine candidates for SARS-COV-2.

Considering what is already revealed by the state of the art and that the present application relates to a new broad-action platform, allowing the development of vaccines against different diseases, such as COVID-19, Influenza, dengue, malaria and that leishmaniasis it presents new polynucleotide constructs, it can be considered that the present application does not become obvious, since it is not possible to predict it, and, mainly, guarantee its functionality through the documents revealed by the state of the art.

### SUMMARY OF THE INVENTION

In order to overcome the problems cited above, the present invention will provide significant advantages over the development of plasmids to be used in an existing DNA vaccine platform for SARS-COV-2 and/or Influenza, enabling an increase in its performance and presenting a more favorable cost/benefit ratio.

The present application refers to the development of a vaccine platform consisting of two plasmids pCTV1 and pCTV2, produced from the modification of a commercial plasmid (pCDNA 3.1) in a way that only the genes which are essential to the expression of proteins in mammal cells were maintained, namely:
a. CMV promoter and enhancer—Strong promoter of Human Cytomegalovirus
b. SV40 promoter and enhancer—Simian vacuolating virus 40 promoter (only in pCTV2)
c. Multiple Cloning Site (MCS)—Insertion site of the gene of interest
d. Bovine Growth Hormone polyadenylation signal (beta subunit)
e. High-copy replication origin (ColE1)
f. Ampicillin and/or Kanamycin Resistance Gene Coupled to the Respective Promoter.

The generated plasmids have about 2,000 pb less than the commercial plasmid having approximately ˜5.4 kb, moving to ˜3.0 kb (pCTV1) and ˜3.4 kb (pCTV2).

The present platform can be used in the development of vaccines for different diseases such as, for example, COVID-19, seasonal influenza, dengue, malaria and leishmaniasis. In the present application, the plasmid pCTV1 has already been assessed as a vaccine component against COVID-19 and influenza, which carries the gene sequence encoding the SARS-COV-2 Spike protein (pCTV-WS) and/or which carries the gene sequence encoding the Influenza NP protein (pCTV1-NP).

The present application refers to one vaccine using a plasmid DNA comprising the sequence encoding the S protein of the original Wuhan SARS-COV-2 (pCTV-WS); the sequence encoding the Influenza NP protein (pCTV1-NP), the H1 protein encoding the Influenza H1 protein (pCTV-H1) and the sequence encoding the SARS-COV-2 S protein and the Influenza NP protein (pCTV2-NP-Spike); and the efficacy of pCTV-WS against the Wuhan isolate and VOCs of SARS-COV-2 was evaluated in the transgenic mouse model (K18-hACE2) and hamster, who develop disease that resembles the severe and moderate forms seen in humans, respectively. The results show that this vaccine induces high, intermediate, and low levels of nAbs to the Wuhan, Delta, and Gamma isolate of SARS-COV-2, respectively, but no nAbs to the Omicron variant of SARS-COV-2. Immunization with the pCTV-WS vaccine also induced a robust response from T cells to the WS protein. Protective immunity induced by this vaccine was mediated primarily by nAbs and T cells for Wuhan/Delta and Gamma/Omicron, respectively. As regards the Influenza virus, high levels of total IgG, IgG1 and IgG2c antibodies and also of hemagglutination inhibitor antibodies in animals immunized with pCTV1-NP.

The invention further relates to recombinant plasmid pCTV1 which carries the gene sequence encoding the SARS-CoV-2 Spike protein (pCTV-WS), as well as its immunogenic profile and ability to prevent high viral loads and moderate and severe clinical forms of the disease through stimulation of the immune system through immunization, against the ancestral isolate of SARS-COV-2 and against different variants of concern.

The invention further relates to recombinant plasmid pCTV1 which carries the gene sequence encoding the NP protein, of the PR8 isolate, with the ability to prevent high viral loads and moderate and severe clinical forms of the disease through stimulation of the immune system through immunization, facing the challenge with the A/H1/Brisbane/2018 (H1N1) and A/H3/South Australia (H3N1) isolates.

In a second aspect, the present invention relates to the development of a vaccine platform consisting of plasmid pCTV2, produced in the same manner as plasmid pCTV1. However, the pCTV2 was generated to allow the insertion of two genes in a single plasmid using two independent promoters (SV40 and CMV).

The invention further relates to recombinant plasmid pCTV2 which carries the gene sequences encoding the NP and Spike proteins.

Both generated plasmids became more efficient for the expression of proteins of interest for vaccines.

The present platform can be used in the development of vaccines for different diseases such as COVID-19, seasonal Influenza, dengue, malaria and leishmaniasis.

In a third aspect, the invention further relates immunogenic composition consisting of recombinant to a DNA plasmid pCTV1 which carries the gene sequence encoding the Spike protein, with the ability to prevent high viral loads and moderate and severe clinical forms of the disease through stimulation of the immune system through immunization, against both the ancestral isolate of SARS-COV-2 and different variants of concern.

In a fourth aspect, the invention further relates to a DNA immunogenic composition consisting of the recombinant plasmid pCTV1 which carries the gene sequence encoding the NP protein, of the PR8 isolate, with the ability to prevent high viral loads and moderate and severe clinical forms of the disease through stimulation of the immune system through immunization, facing the challenge with A/H1/Brisbane/2018 (H1N1) and A/H3/South Australia (H3N1) isolates.

In a fifth aspect the invention further relates to a bivalent DNA immunogenic composition consisting of the recombinant plasmid pCTV2 which carries the gene sequences encoding the NP and Spike proteins.

In a sixth aspect the present application refers to the use of the plasmids and of the immunological compositions for the preparation of an immunobiological drug with the ability to prevent high viral loads and moderate and severe clinical forms of the disease through stimulation of the immune system through immunization, both against Influenza and SARS-COV-2 strains and their variants of concern.

### BRIEF DESCRIPTION OF THE DRAWINGS

The structure and operation of the present patent application, together with additional advantages thereof can be better understood by reference to the attached drawings and the following description:

FIG. 1 illustrates the base Plasmid (pCTV1);

FIG. 2 illustrates the Plasmid for the bivalent vaccine-two promoters (pCTV2);

FIG. 3 (panel A-panel D) shows the Plasmid pCTV1-H1 (A/Brisbane/18).

FIG. 4 (panel A-panel C) shows the Plasmid pCTV1-NP (PR8).

FIG. 5 shows an Immunofluorescence of HEK 293T cells transfected with the plasmid pCTV1-NP (PR8).

FIG. 6 (panel A-panel F) shows the evaluation of the immune response of animals vaccinated with pCTV1-NP (PR8).

FIG. 7 (panel A-panel H) shows the protection against the isolates of A/H1/Brisbane/02/18 (H1N1) and A/H3/South Australia/34/19 (H3N1) influenza viruses conferred by immunization with the pCTV1-NP (PR8);

FIG. 8 shows the co-expression of the fluorescent proteins GFP and RFP in HEK cells transfected with the plasmid pCTV2 encoding fluorescent proteins (GFP and RFP);

FIG. 9 shows the co-expression of Spike (green) and NP (red) proteins in HEK cells transfected with the plasmid pCTV2 encoding the Spike (SARS-COV-2) and NP (Influenza) proteins;

FIG. 10 shows the in vitro expression of Spike and NP proteins in HEK cells transfected with the plasmid pCTV2 encoding Spike (SARS-COV-2) and NP (Influenza) proteins;

FIG. 11 (panel a-panel e) shows the generation of the plasmid pCTV-WSpike (pCTV-WS);

FIG. 12 (panel a-panel g) shows the immunogenicity of the pCTV-WS in K18-hACE2 immunized mice;

FIG. 13 (panel a-panel h) shows the protection, viral load, and histopathology in K18-hACE2 mice immunized with pCTV-WS and challenged with the Wuhan isolate;

FIG. 14 (panel a-panel j) shows the inflammatory and immunohistochemical profile of B and T cells in lung samples of K18-hACE2 mice immunized with pCTV-WS and challenged with the Wuhan isolate;

FIG. 15 (panel a-panel q) shows the titer of nAbs and protection against SARS-COV-2 variants of concern in K18-hACE2 mice immunized with pCTV-WS and challenged with the Delta, Gamma, and Omicron variants;

FIG. 16 (panel a-panel i) shows the immunogenicity and protection of pCTV-WS against the Wuhan isolate in the Syrian hamster model;

FIG. 17 (panel a-panel o) shows nAb titers and protection against SARS-COV-2 variants of concern in Syrian hamsters immunized with pCTV-WS and challenged with the Delta, Gamma, and Omicron variants; and

FIG. 18 (panel a-panel x) shows the immunological mechanisms involved in the protective immunity of K18-hACE2 mice immunized with pCTV-WS and challenged with the Wuhan isolate and the Delta, Gamma, and Omicron variants.

### DETAILED DESCRIPTION OF THE INVENTION

Before the present invention is described in more detail, it should be understood that this invention is not limited to the particular embodiments described, as such may vary. It should also be understood that the terminology used herein is solely for the purpose of describing particular embodiments and is not intended to be limiting, as the scope of the present invention will be limited only by the appended claims.

As will be apparent to those skilled in the art after reading this disclosure, each of the individual embodiments described and illustrated herein has discrete components and features that can be readily separated or combined with the features of any of the other several embodiments without departing from the scope or spirit of the present invention. Any method recited may be performed in the order of the recited events or in any other order that is logically possible.

The present application refers to the development of a vaccine platform consisting of two plasmids pCTV1 and pCTV2, produced from the modification of a commercial plasmid (pCDNA 3.1) so that only the genes essential for the expression of proteins in mammalian cells were maintained, namely: a. CMV promoter and enhancer-Human Cytomegalovirus strong promoter and SV40 promoter and enhancer-Simian vacuolating virus 40 promoter (only in pCTV2) b. Multiple Cloning Site (MCS)—Insertion site of the gene of interest c. Bovine Growth Hormone polyadenylation signal (beta subunit) d. High-copy origin of replication (ColE1) e. Ampicillin Resistance Gene coupled to its respective promoter.

The present platform can be used in the development of vaccines for different diseases such as, COVID-19, seasonal Influenza, dengue, malaria and leishmaniasis. In the present application, the plasmid pCTV1 has already been assessed as a vaccine component against COVID-19, which carries the gene sequence encoding the Spike protein of the Wuhan isolate of the SARS-COV-2 (pCTV-WS). F Furthermore, the pCTV1 which carries the genetic sequence encoding the NP protein of the PR8 isolate of the influenza virus has also been assessed.

The present application refers to a vaccine using a plasmid DNA comprising the sequence encoding the SARS-COV-2 Spike protein of the Wuhan isolate (pCTV-WS) and tested the effectiveness of immunization against the Wuhan isolate and VOCs in transgenic mouse (K18-hACE2) and hamster model, who develop disease that resembles the severe and moderate human form, respectively. The results show that this vaccine induces high, intermediate, and low levels of nAbs to Wuhan, Delta, and Gamma, respectively, but no nAbs for the Omicron variant. The immunization with the pCTV-WS vaccine also induced a robust response from T cells to the WS protein. The protective immunity induced by this vaccine was mediate primarily by nAbs and T cells for Wuhan/Delta and Gamma/Omicron, respectively.

### EMBODIMENTS OF THE PRESENT INVENTION

### Material and Methods

### Vaccine Vectors Construction

According to the resolution of the Food and Drug Administration—FDA—entitled Considerations on Plasmid DNA Vaccines Indicated for Infectious Diseases, DNA vaccines are understood as preparations of plasmids containing one or more DNA sequences, capable of inducing and/or promoting an immune response against a pathogen. In general, these plasmids contain: 1) DNA sequences required for selection and replication in bacteria (i.e., resistance genes and origin of replication); 2) sequences that promote gene expression in vaccine recipients (i.e., eukaryotic promoters, enhancers, terminators, and polyadenylation signals); 3) may also contain immunomodulatory elements.

Base Plasmid (pCTV1)—SEQ ID NO. 1:

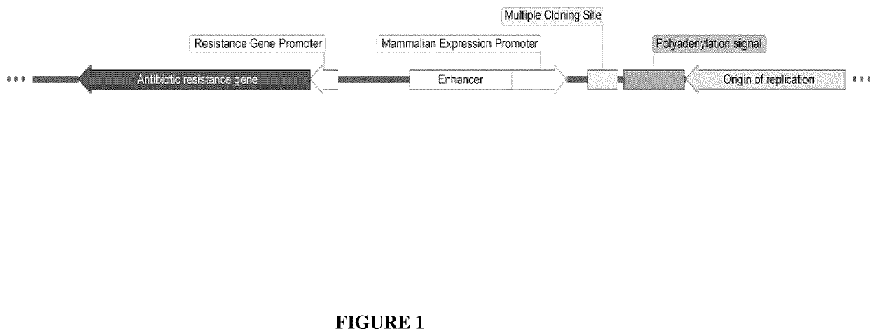

The plasmid pCTV1 (FIG. 1) was developed from the commercial plasmid which is a known vector for expression in eukaryotic cells. The main modification was to reduce the size of the backbone of the plasmid, maintaining only the strictly essential elements for growth in bacteria, expression in mammalian cells and ectopic gene expression:
a. Promoter and enhancer for expression in human cells
b. Multiple Cloning Site-Insertion site of the gene of interest
c. Polyadenylation signal
d. High-copy origin of replication
e. Antibiotic resistance gene coupled to the respective promoter. Kanamycin resistance gene will be used, as this antibiotic is used less in medical practice.
   Plasmid for the Bivalent Vaccine-Two Promoters (pCTV2)—SEQ ID NO. 2:

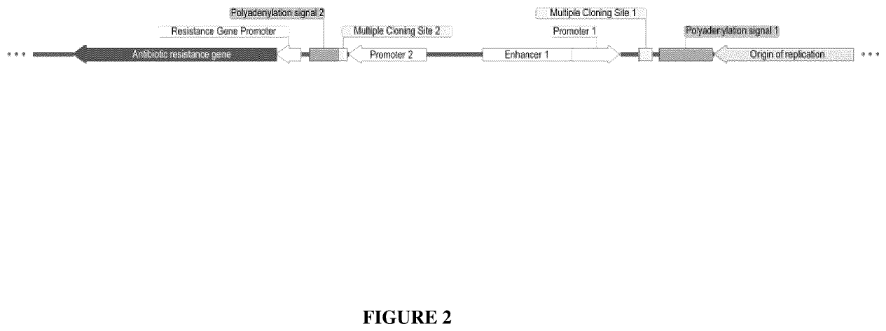

The objective of the plasmid pCTV2 (FIG. 2) is to allow the insertion of two distinct antigenic sequences [for example, Nucleoprotein (Influenza) and Spike (SARS-COV-2)] under the regulation of another expression promoter in mammalian cells. Both promoters are among the most widely used for this purpose. In addition to the insertion of the second set (promoter+enhancer+polyadenylation signal), it was necessary to construct a new multiple cloning site at the expense of reducing the first site, to avoid the repetition of restriction sites, making enzymatic digestions for cloning unfeasible. Additional elements of the plasmid pCTV2:
a. SV40 promoter and enhancer-Simian vacuolating virus 40 promoter
b. MCS 2-Multiple Cloning Site 2-Insertion site of the second gene of interest
c. SV40 polyadenylation signal

### In Vitro Expression of Viral Proteins

To evaluate the expression of proteins of interest by immunofluorescence, monolayers of HEK293T cells were transfected with the pCTV-WS (SEQ ID NO. 4), pCTV1-H1 (SEQ ID NO. 7) or pCTV1-NP (SEQ ID NO. 3) plasmids. After 48 hours the cells were fixed and stained. The slides were observed by confocal microscopy. Similarly, for detection of expression by western blot, HEK293T cells were transfected and after 48 hours the cell extract was collected and after electrophoresis, the proteins were transferred to nitrocellulose membrane and incubated with the respective antibodies.

### Immunization of C57BL/6 Mice and Evaluation of Humoral and Cellular Response

For the immunization female mice of the C57BL/6 lineage, 6 to 8 weeks old, were used, which were obtained from the Production Animal Facility of the René Rachou Institute. The animals were handled in accordance with CONCEA regulations and according to the protocol approved by the Ethics Committee on the Use of Laboratory Animals (CEUA LW25/20-FIOCRUZ).

A series of homologous priming/boosting protocols using 100 μg of plasmid DNA intramuscularly were assessed:
pCTV1-NP/pCTV1-NP (SEQ ID NO. 3)
pCTV1-WS (SEQ ID NO. 4)/pCTV1-WS
pCTV2-NP-Spike (SEQ ID NO. 5)/pCTV2-NP-Spike

Mice were immunized using the protocols described above and as control, mice immunized with pCTV1 without the insert of interest (pCTVØ), or saline solution (PBS) were used. Immunizations were performed with 100 μg of the plasmid of interest diluted in saline solution (vehicle), intramuscularly. Eighteen days after the initial dose (prime), a blood sample was collected from the animals to assess the humoral response. Twenty-one days after the first dose, the booster dose was administered. Another 30 days after the booster dose, another serum collection was performed, with subsequent euthanasia of the animals and collection of the spleen and bronchoalveolar lavage fluid (BALF). The sera and BALF collected were used to analyze the humoral response, with the spleen being used to evaluate the cellular immune response induced by immunization.

### Enzyme-Linked Immunosorbent Assay—ELISA

96-well plates were sensitized with recombinant proteins of interest (Spike and NP). The immunized mice sera were diluted and used as primary antibody. Anti-IgG antibody conjugated to peroxidase was used as secondary antibody. The ELISA was developed with chromogenic substrate and the plates were read at 450 nm.

### Determination of IFN-γ in Spleen Cell Culture Supernatants

C57BL/6 mice were immunized as previously described. Four weeks after the last immunization, the animals were euthanized for spleen collection. Then, the splenocytes were isolated and restimulated with 10 μg of the protein used as the gene of interest. After 72 hours of stimulation, the culture supernatant was collected to determine IFN-γ production. IFN-γ quantification was performed by ELISA, and the cytokine concentration in each sample was determined from a standard curve.

### Results

### Plasmids Construction

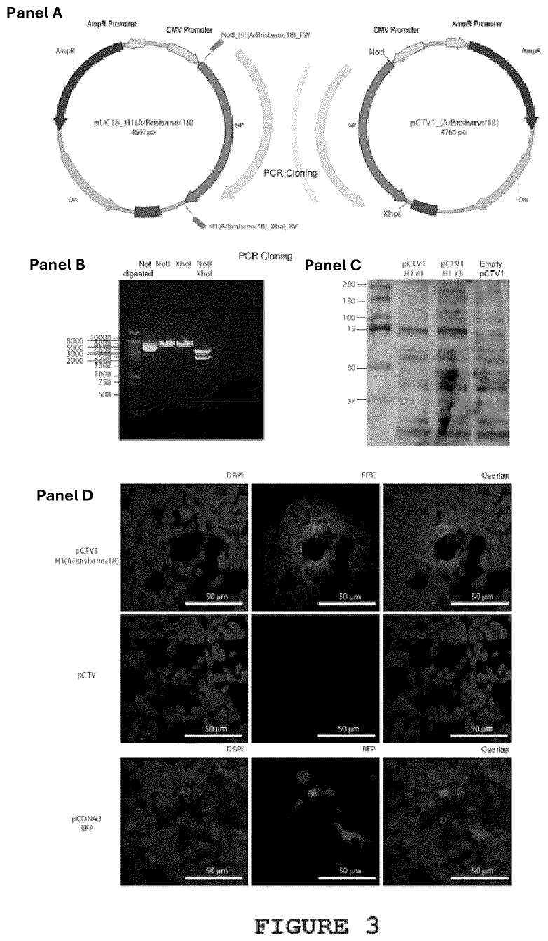

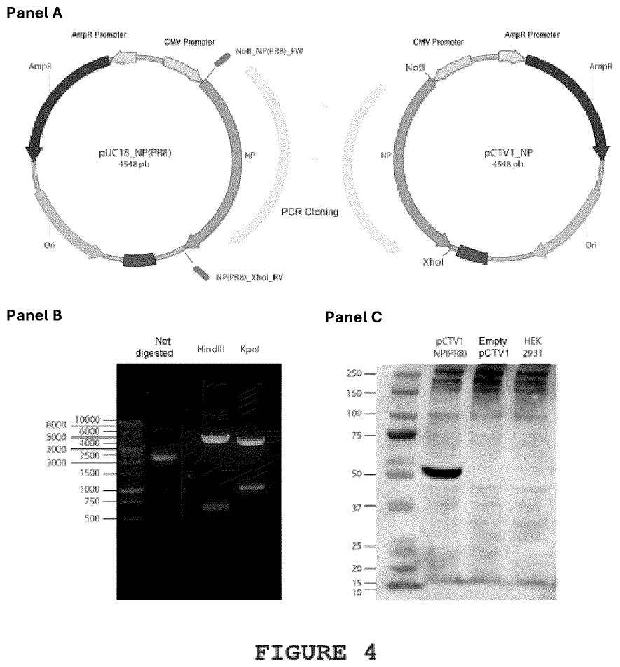

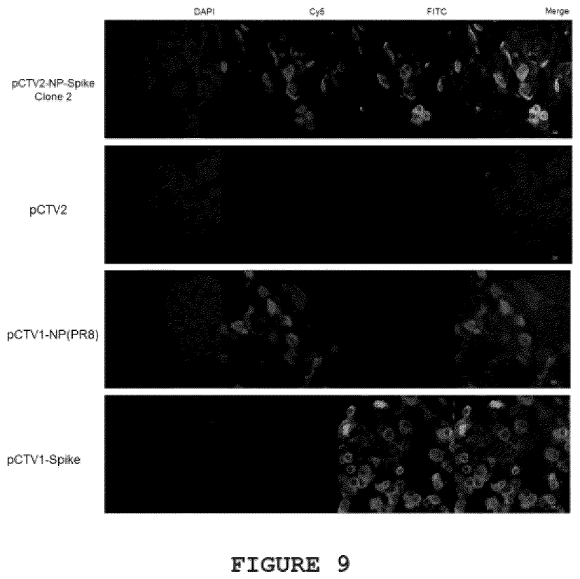

Initially the success of the construction of the plasmids pCTV-WS (SEQ ID NO. 4), pCTV1-H1 (SEQ ID NO. 7), pCTV1-NP (SEQ ID NO. 3) and pCTV2-NP-Spike (SEQ ID NO. 5) was verified by enzymatic digestion with restriction enzymes to release the insert of interest. Complete sequencing of the plasmids confirmed the absence of mutations in the gene sequences. Furthermore, after transfection of permissible cells it was possible to detect the expression of the Spike (FIGS. 9, 10 and 11), H1 (FIG. 3) and NP (FIGS. 4, 5, 9 and 10) proteins, both by immunofluorescence and by western blot.

### Immune Response Assessment

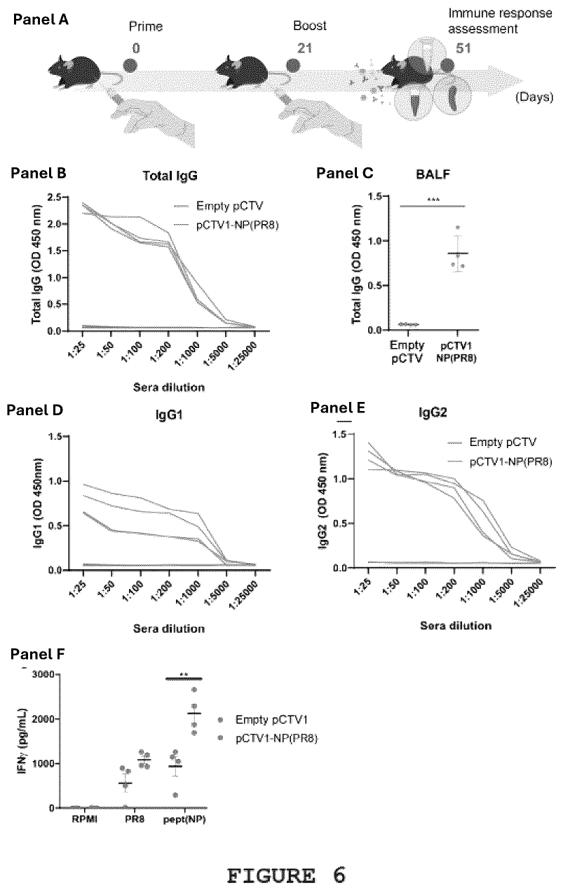

Given the confirmation of the expression of the genes of interest using plasmid DNAs, female mice, 6-8 weeks old, were immunized with the plasmids pCTV-WS and pCTV1-NP. Thirty days after the last vaccination, high levels of total IgG antibodies and subclasses specific to the proteins used as target genes were detected in serum samples from the animals (FIGS. 6, 12 and 16). The production of total IgG in the bronchoalveolar lavage fluid (BALF) of these animals was also significant when compared to the control group. Finally, the measurement of IFN-γ in the culture supernatant of splenocytes restimulated in vitro with recombinant proteins demonstrated that immunization was capable of inducing the generation of memory T cells for the proteins used as the gene of interest (FIGS. 6, 12 and 16).

Therefore, immunization with the developed plasmids, pCTV-WS and pCTV1-NP, is capable of inducing both humoral and cellular responses, the next steps consisted of immunization, followed by challenge with different isolates of the influenza virus and SARS-COV-2 to evaluate the protection conferred.

FIG. 3 shows Plasmid pCTV1-H1 (A/Brisbane/18). (Panel A) Schematic drawing of the construction of the pCTV1-H1 (A/Brisbane/18) plasmid. (panel B) Confirmatory digestion with NotI (4766 bp), XhoI (4766 bp) and release of the insert with simultaneous digestion with NotI and XhoI (3008pb and 1701 pb corresponding to pCTV1 and H1 (A/Brisbane/18) respectively. (Panel C) Western blot of HEK293T cells transfected with pCTV1-H1 (A/Brisbane/18) after 72 hours of transfection, the protein extract from the cells was collected for running on a polyacrylamide gel. After transfer, the proteins were incubated with serum from mice immunized with the influenza virus HA (A/Brisbane/18) to detect the H1 protein (72 kDa). (Panel D) HEK293T cells were transfected and after 72 hours were primarily labeled with anti-H1 (A/Brisbane/18) polyclonal antibody to detect the protein.

FIG. 4 shows Plasmid pCTV1-NP (PR8). (panel A) Schematic drawing of the construction of the pCTV1-PR8 plasmid. (Panel B) Confirmatory digestion with HindIII (3496 and 601pb), KpnI (3181 and 916pb). (panel C) Western blot of HEK293T cells transfected with pCTV1-NP (PR8)—After 72 hours of transfection, the protein extract from the cells was collected for running on polyacrylamide gel. After transfer, the proteins were incubated with serum from mice immunized with the Influenza A/PR/8/34 virus for NP protein detection (62 kDa).

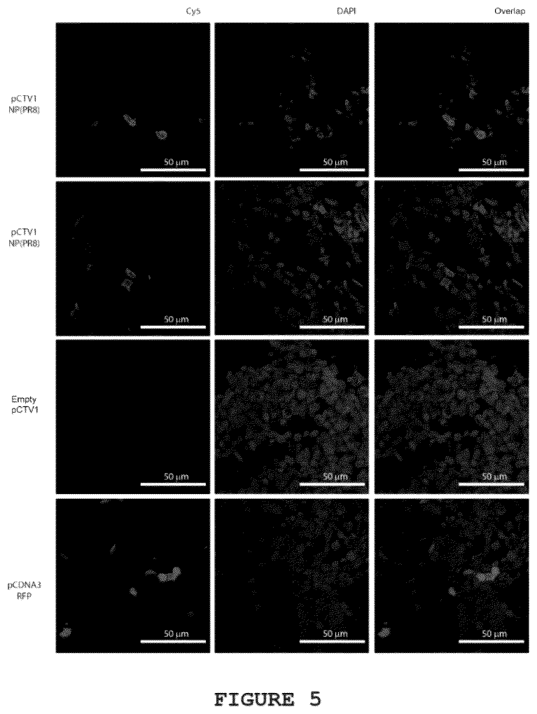

FIG. 5 shows the Immunofluorescence of HEK293T cells with the plasmid pCTV1-NP (PR8). After 48 hours of transfection, the cells were incubated with mouse serum immunized with the Influenza A/PR/8/34 virus and labeled with anti-mouse secondary antibody Cy5 to identify the expression of the NP protein.

FIG. 6 shows the evaluation of the immune response of animals vaccinated with pCTV1-NP (PR8). Immunization scheme (panel A) Groups of 4 mice received two doses of the plasmid intramuscularly with an interval of 21 days and were euthanized after 30 days of the complete vaccination for collection of blood (serum) and bronchoalveolar lavage (BALF), for antibody measurement and the spleen for measurement of IFN-γ in the supernatant of the splenocyte culture of the immunized animals. (Panels B, D and E) The immunization with the plasmid pCTV1-NP (PR8) was able to induce the production of antibodies, total IgG, and subclasses (IgG1 and IgG2), against the A/PR/8/34 virus. (Panel C) Likewise, it was also possible to detect the presence of total IgG in BALF samples from immunized animals. Finally, the IFN-γ production in the supernatant of the culture of splenocytes from mice immunized after stimulation with total proteins of the A/PR/8/34 virus and with the pool of nucleoprotein (NP) peptides.

### Challenge Essay

Groups of 4 female C57BL/6 mice were immunized with two doses of pCTV1-NP (PR8) or pCTV-Ø (control) 21 days apart. Thirty days after completing the immunization protocol, the animals were challenged with a lethal dose (2×10⁴ PFU) of recombinant viruses expressing seasonal H1 (A/H1/Brisbane/02/18) or H3 (A/H3/South Australia/34/19). Weight loss and survival were monitored for 12 days, and the animals were euthanized for collection of blood and lung samples.

### Lung Viral Load

The sectioned portion of the collected lung was macerated in sterile 1× PBS in the tissue homogenizer. After centrifugation, the supernatant was successively diluted for titration in MDCK cell monolayers by lysis plate under agarose.

### Hemagglutination Inhibition (HAI)

Briefly, the collected sera were successively diluted, and the dilutions were incubated with 4 hemagglutinating units of the virus used in the challenge. At the end of incubation, a suspension of red blood cells was added, so that the hemagglutination inhibition titer was determined by the formation of the red blood cell button, which only decants in the presence of antibodies capable of preventing the interaction of the viral particle with the red blood cells, otherwise, in the absence of antibodies, the viral particle is able to promote the hemagglutination of red blood cells and they remain diffuse, without the formation of the button.

### Description and Analysis of Results

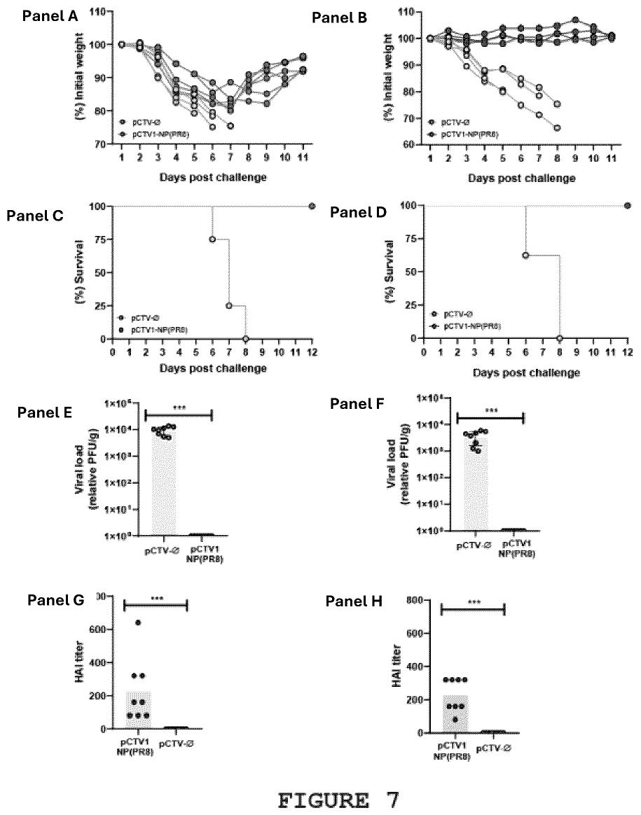

The results demonstrated that the immunization with pCTV1-NP (PR8) confers protection to animals against influenza viruses expressing different seasonal HAs (H1 and H3), so that animals that received the empty vaccine vector (pCTV-Ø) succumbed to infection (FIG. 7 panels A, B, C and D). Furthermore, the immunization with the pCTV1-NP (PR8) also promoted a reduction in viral load in the lungs of mice compared to the control group (pCTV-Ø) (FIG. 7—panel E and panel F). Finally, corroborating the results described previously, only animals immunized with pCTV1-NP (PR8) showed hemagglutination inhibition titers against the virus that were challenged (FIG. 7—panel G and panel H).

FIG. 7 shows protection against influenza viruses which carries the A/H1/Brisbane/02/18 or A/H3/South Australia/34/19 conferred by the immunization with pCTV1-NP (PR8). Weight loss and survival (panel A and panel C), viral load in the lung (panel E) and hemagglutination inhibition (panel G) using serum from mice immunized with pCTV1-NP (PR8) (green circle) or pCTV-Ø (grey circle) and challenged with the influenza virus which carries H1. Weight loss and survival (panel B and panel D), viral load in the lung (panel F) and hemagglutination inhibition (panel H) using serum from mice immunized with pCTV1-NP (PR8) (pink circle) or pCTV-Ø (grey circle) and challenged with the influenza virus which carries H3. The data presented were obtained from two independent experiments. The statistical analyses used the Mann-Whitney test. Bars represent the mean. *** p<0,0001.

pCTV2 Plasmid

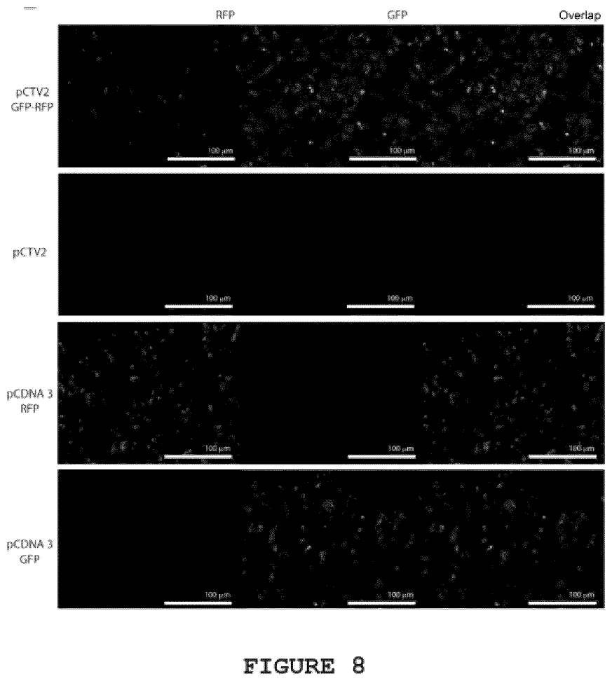

The pCTV2 plasmid has two multiple cloning sites, which allows the simultaneous insertion of two genes of interest. The pCTV2 co-expressing Red Fluorescent Protein (RFP) and Green Fluorescent Protein (GFP) (FIG. 8—SEQ ID NO. 6) was produced for use in the present invention. As can be seen in the immunofluorescence images, in the upper right panel, the cells co-expressing the RFP and GFP proteins show orange staining.

FIG. 8 shows the co-expression of the fluorescent proteins GFP and RFP in HEK cells transfected with the plasmid pCTV2 encoding fluorescent proteins (GFP and RFP). Fluorescence microscopy demonstrating the co-expression of the GFP and RFP proteins by pCTV2 (upper panel), orange cells (overlay) demonstrate the co-expression of the two proteins. As controls, the plasmid pCTV2 without insert (second panel), pCDNA3 RFP (third panel) and pCDNA3 GFP (last panel) were used.

The plasmid pCTV2 was also used for the generation of the bivalent DNA vaccine for COVID-19 and seasonal influenza. The genes encoding the Spike (SARS-COV-2) and NP (Influenza) proteins were inserted. As can be seen in the immunofluorescence images in FIG. 9, the Spike protein was stained green, and the NP protein was stained red. Cells co-expressing both proteins (Spike and NP) are stained orange (upper panel-right).

FIG. 9 shows co-expression of Spike (green) and NP (red) proteins in HEK cells transfected with the pCTV2 plasmid encoding Spike (SARS-COV-2) and NP (Influenza) proteins. Fluorescence microscopy demonstrating co-expression of Spike and NP proteins by pCTV2 (upper panel), orange cells (overlay) demonstrate co-expression of both proteins. As controls, plasmids pCTV2 without insert (second panel), pCTV1-NP (third panel) and pCTV1-WS (last panel) were used.

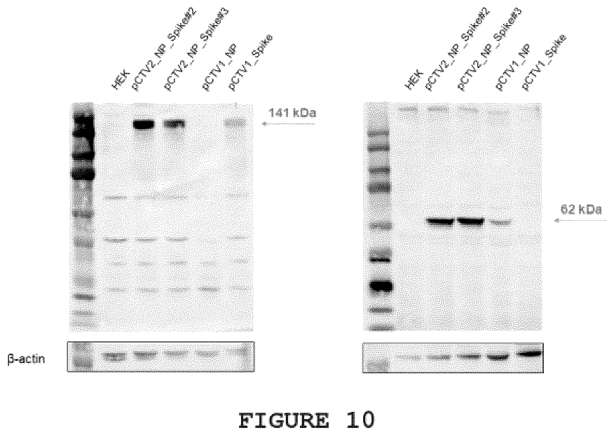

The expression of Spike and NP proteins in vitro was also evaluated by western blot. As shown in FIG. 10, HEK293A cells transfected with pCTV2-NP-Spike (SEQ ID NO. 5) were able to synthesize Spike (left image) and NP (right image) proteins.

FIG. 10 shows an in vitro expression of Spike and NP proteins in HEK cells transfected with plasmid pCTV2 (SEQ ID NO. 5) encoding Spike (SARS-COV-2) and NP (Influenza) proteins. Western blot demonstrating the expression of Spike (left image) and NP (right image) proteins by pCTV2-NP-Spike. As controls, untransfected cells (HEK), pCTV1 expressing only NP protein (pCTV1-NP) and pCTV1 expressing only Spike protein (pCTV1-WS) were used. β-actin: endogenous control. Spike: ˜ 141 kDa. NP: ˜ 62 kDa.

### Definitions

The term “pharmaceutically acceptable vehicle” as used herein means any material or substance with which the active ingredient is formulated to facilitate its application or dissemination to the site to be treated, for example, by dissolving, dispersing or diffusing the composition, and/or to facilitate its storage, transportation or handling without impairing its efficacy. They include any and all solvents, dispersion media, coatings, antibacterial and antifungal agents (for example, phenol, sorbic acid, chlorobutanol), isotonic agents (for example, sugars or sodium chloride) and the like. Additional ingredients may be included to control the duration of action of the immunogenic peptide in the composition. Suitable pharmaceutical carriers for use in the pharmaceutical compositions and their formulation are well known to those skilled in the art and there is no particular restriction on their selection in the present invention. They may also include additives such as wetting agents, dispersing agents, adhesives, emulsifying agents, solvents, coatings, antibacterial and phenol, antifungal agents (for example, sorbic acid, chlorobutanol), isotonic agents (such as sugars or sodium chloride) and the like, provided that they are consistent with pharmaceutical practice, i.e., carriers and additives that do not create permanent harm to humans.

The immunological compositions of the present invention may be prepared in any known manner, for example by homogeneously mixing, coating and/or grinding the active ingredients, in a single or multi-step procedure, with the selected carrier material and, where appropriate, the other additives.

The immunological composition should comprise an immunologically effective amount of the active ingredient as indicated below in relation to the method of treatment or prevention. Optionally, the composition further comprises other therapeutic ingredients. Other suitable therapeutic ingredients, as well as their usual dosage depending on the class to which they belong, are well known to those skilled in the art and may be selected from other known drugs used to treat immune disorders.

The term “immunologically effective amount” refers to the amount that reduces, to some extent, one or more symptoms of the disease or disorder, and, more particularly, returns to normal, partially, or completely, the physiological or biochemical parameters associated with or causing the disease or disorder. Typically, the immunologically effective amount is the amount that will lead to an improvement or restoration of the normal physiological situation.

### Methods

Current COVID-19 vaccines protect against severe disease but are not effective in controlling the replication of Variants of Concern (VOCs).

In the present application, existing preclinical models of severe and moderate COVID-19 were used to evaluate the efficacy of a DNA vaccine based on Spike (pCTV-WS) for protection against different VOCs. The immunization of transgenic mice (K18-hACE2) and hamsters induced significant levels of neutralizing antibodies (nAbs) for Wuhan isolates and Delta variant, but not for Gamma and Omicron variants. However, the pCTV-WS vaccine offered significant protection to all VOCs. Consistently, protection against lung pathology and viral load to Wuhan or Delta was mediated by nAbs, while in the absence of nAbs, T cells significantly controlled viral replication, disease and lethality in mice infected with the Gamma or Omicron variants. Thus, considering the conserved nature of the epitopes of CD4 and CD8 T cells, the present application corroborates the hypothesis that the induction of effector T cells should be a primary objective for new vaccines against emerging VOCs SARS-CoV-2.

Data from the literature has highlighted the importance of T cells in mediating immunity to SARS-COV-2, and its VOCs Alpha, Beta, Delta, and Omicron. Individuals vaccinated with adenovirus or mRNA vaccine and convalescents from COVID-19 have low levels of neutralizing antibodies (nAbs), but a robust cross-reactivity of CD4 and CD8 T cells against VOCs, including the Omicron variant. These findings suggest that protection against severe disease, despite low levels of VOC-specific nAbs, is largely mediated by T cells.

In this context, a DNA-based platform that induces a strong protective T cell-mediated immune response is an important strategy to rapidly adapt COVID-19 vaccines to new VOCs.

The inventors of the present application tested an experimental vaccine using plasmid DNA comprising the coding sequence of the original SARS-COV-2 Spike protein from Wuhan (pCTV-WS) and assessed the effectiveness of the vaccination against SARS-COV-2 VOCs in experimental transgenic mouse (K18-hACE2) and hamster models, who develop severe and moderate disease similar to human disease, respectively. The results showed that such vaccine induced high, intermediate, and low levels of nAbs for the Wuhan isolate and the Delta and Gamma variants, respectively, but no nAbs against the Omicron variant. The immunization with the pCTV-WS vaccine also induced a robust response of memory T cells. The protective immunity induced by this vaccine was mediated mainly by nAbs and T cells for Wuhan/Delta and Gamma/Omicron, respectively.

### Study Design

The aim of the present study was to evaluate the immunogenicity and efficacy of a plasmid DNA-based COVID-19 vaccine against the original SARS-COV-2 and variants of concern in experimental models of the disease (K18-hACE2 mice and hamsters). To this end, a plasmid containing the complete sequence of the gene encoding the Spike protein of the original (Wuhan) strain of SARS-COV-2 (pCTV-WS) (SEQ ID NO. 4) was constructed. The expression of the Spike gene sequence, cloned in the pCTV vector, was confirmed by Western blot and immunofluorescence techniques. The inventors of the present application immunized transgenic K18-hACE2 mice and hamsters with pCTV-WS and evaluated humoral and cellular immune responses by analyzing the levels of neutralizing and non-neutralizing antibodies, frequency of different immune cell subpopulations and cytokine and chemokine levels in lung tissue. Techniques that were also used are serum transfer, T cells depletion and adoptive transfer of T cells to evaluate the mechanisms of immunological protection induced by the experimental vaccine pCTV-WS. The immunized animals were also challenged with the original SARS-COV-2 isolate (Wuhan) and the Delta, Gamma, and Omicron variants of concern in a biosafety level 3 laboratory to assess the level of protection, evaluating body weight, survival or viral load/viral RNA in lung samples and nasal wash. At the end of the experiments, the animals were euthanized for histopathological evaluation of lung tissue.

### Ethics Statement and Experimental Models

All animal experiments were performed according to the principles of conduct of the Brazilian Guide of Practices for the Care and Use of Animals for Scientific and Educational Purposes of CONCEA (http://www.sbcal.org.br/). The animal experimentation protocols were approved by the Animal Use Ethics Committee (CEUA) of the Oswaldo Cruz Foundation (CEUA protocol LW25/20) and the University of São Paulo (CEUA protocol 105/2020). Female WT C57BL/6 mice, 6-10 weeks old, were purchased from the Animal Facility of the Federal University of Minas Gerais (CEBIO-UFMG). Transgenic mice expressing human angiotensin-converting enzyme (K18-hACE2) in the C57BL/6 background, 6-10 weeks old, were originally obtained from Jackson's Laboratory and were bred in the Production Facility of Fiocruz-Minas. Transgenic mice expressing human angiotensin-converting enzyme and deficient in B cells (K18-hACE2/B-KO) on the C57BL/6 background, 6-10 weeks old, were bred at the Ribeirão Preto School of Medicine (USP). Golden Syrian hamsters, 6-10 weeks old, were obtained from Fiocruz-Minas and used as a model of moderate COVID-19.

The present invention was registered in the SisGen-Sistema Nacional de Gestão do Patrimônio Genético e do Conhecimento Tradicional Associado (National Management System for Genetic Heritage and Associated Traditional Knowledge, in English) with Access Registration No. A114B6C, in compliance with the provisions of Law No. 13.123/2015 and its regulations.

### Animal Immunization and Challenge Experiments

C57BL/6, K18-hACE2 or K18-hACE2/B-KO mice and hamsters received two doses containing 100 μg of pCTVØ (mock immunization) or pCTV-WS diluted in PBS, 21 days apart. The solution was administered intramuscularly in a final volume of 50 μl in each hind paw (tibialis muscle). Thirty days after the second dose, K18-hACE2 or K18-hACE2/B-KO mice were challenged intranasally with 5×10⁴ PFU of SARS-COV-2 (for the Wuhan strain, Delta, and Omicron variants) and 5×10³ PFU of SARS-CoV-2 (for the Gamma variant). The hamsters were challenged with 1×10⁵ PFU of SARS-COV-2 (for the Wuhan strain, Delta, and Omicron variants) and 1×10⁴ PFU of SARS-COV-2 (for the Gamma variant). Body weight, clinical signs and survival were monitored daily after infection.

### CD4+/CD8+ T Cell Depletion, Serum Transfer and Adoptive Transfer

K18-hACE2 mice immunized with pCTV-WS were treated with 0.5 mg/mouse of rat anti-mouse CD4a mAb (clone GK1.5; Cat. #BE0003-1; BioXCell) and 0.5 mg/mouse of rat anti-mouse CD8a mAb (clone 2.43; Cat. #BE0061; BioXCell) or 0.2 mg/mouse of rat anti-mouse anti-KLH IgG (clone LTF-2; Cat. #BP0090; BioXCell). Antibodies were administered i.p. on days-3, −2 and −1 before infection and 7 days after infection. T cell depletion levels in treated animals were measured in blood samples by flow cytometry (FIG. 3—panel a). In the serum transfer assay, unimmunized K18-hACE2 mice received 200 μL of serum i.p. from mice previously immunized with two doses of pCTV-WS one day before infection.

The levels of transferred antibodies were determined using the ELISA assay one day after transfer of the sera. For adoptive transfer of T cells, twelve animals immunized with pCTV-WS (SEQ ID NO. 4) were euthanized 30 days after the booster dose. Spleen samples were collected to obtain splenocytes. T cells were purified using a Pan T cell isolation kit (Miltenyi Biotech) and 5×10⁶ purified T cells were adoptively transferred i.v. into unimmunized K18-hACE2 mice one day before infection. The percentage of purified T cells was determined by flow cytometry.

### Virus and Cells

In this study, the strain SARS-COV-2 WT (BRA/SP02/2020 isolate) and Delta (EPI ISL 2965577), Gamma (EPI ISL 2499748) and Omicron (EPI ISL 7699344) variants, isolated from clinical samples of Brazilian patients, were used. Viruses were used to infect Vero E6 cells (ATCC CRL-1586) grown in Dulbecco's Modified Eagle's Medium (DMEM) (Gibco) supplemented with 1% penicillin/streptomycin (Sigma) and 2% fetal bovine serum (Sigma). After 2 days of infection, the culture supernatant was collected and clarified by centrifugation. The viral stocks obtained were titrated by the plaque forming unit (PFU) method and stored at −80° C.

### Plasmids Construction, Culturing, and Purification

The pCTV plasmid containing the genetic sequence encoding the S protein, used in the immunization of experimental animals, was developed at the Center for Vaccine Technologies (UFMG). The codon-optimized Wuhan SARS-COV-2 S gene sequence was obtained from GenScript, cloned into pCTV, and transformed into competent E. coli DH5α cells (Invitrogen) using the heat shock method. A positive colony was selected and cultured in LB medium containing ampicillin (100 μg/mL) at 37° C. for 16 h. Finally, the bacteria were centrifuged and the plasmid (pCTV—WS-SEQ ID NO. 4) was purified using the QIAGEN Plasmid Plus Giga Kit, according to the manufacturer's instructions.

### Western Blot

HEK-293 cells (ATCC CRL-1573) were transfected with 10 μg of pCTV-WS plasmid using Fugene® transfection reagent (Promega). After 72 hours of transfection the cells were harvested and centrifuged. The pellets were resuspended in RIPA buffer (Sigma) comprising protease and phosphatase inhibitor cocktail (complete ULTRA and PhosSTOP, Roche). Protein separation was performed on a 10% SDS-PAGE gel and transferred to a nitrocellulose membrane. The membranes were blocked with 3% bovine serum albumin (BSA) and incubated with primary anti-RBD antibody (Cat. #40592-T62; 1:1000; Sino Biological) or anti-β-actin (Cat. #ab115777; 1:1000; Abcam). Membranes were then incubated with anti-rabbit secondary antibodies (Cat. #A9169; 1:25,000; Sigma) conjugated to HRP and developed using Clarity Max Western ECL substrate (BioRad). Images were captured with an Amersham Imager 600 (GE).

Data were analyzed using ImageJ software (NIH). Blot samples derived from the same experiment were processed in parallel.

### Immunofluorescence

A 4-well slide (Lab-Tek®II) was coated with Vero E6 cells (ATCC CRL-1586) and transfected with 5 μg of pCTV-WS using Fugene® transfection reagent (Promega). Cells were fixed with 4% formaldehyde and permeabilized with PBS+0.3% Triton X-100 (Sigma). The cells were then blocked with 1% BSA and incubated with serum samples from mice immunized with pCTVØ or pCTV-WS at a dilution of 1:200 (v/v). The cells were then incubated with Alexa Fluor 594 goat anti-mouse IgG (Cat. #A-11032; 1:2000; Thermo Fisher). The slides were analyzed under a confocal microscope (Nikon). The images were processed with the NIS-Elements Viewer software (Nikon).

### Detection of Antibodies Specific to Hamster and Mouse Antigens

Plates were coated overnight with 4 μg/mL recombinant WS protein in carbonate buffer and blocked for 2 h with PBS containing 2% bovine serum albumin (PBS-BSA 2%) at 37° C. Serum samples were serially diluted in PBS-BSA 2% and undiluted bronchoalveolar lavage fluid (BALF) samples were applied. After incubation for 1 h at 37° C., plates were washed and then incubated with total anti-mouse IgG (Cat. #1030-05), anti-IgG1 (Cat. #1070-05), anti-IgG2c (Cat. #1079-05) or total anti-hamster IgG (Cat. #6060-05), anti-IgG1 (Cat. #1940-05), anti-IgG2/IgG3 (Cat. #1935-05) conjugated with streptavidin-HRP (1:5000; Southern Biotech). After some washes, the reaction was developed using One-Step TMB (Scienco) for 20 min in the dark, and the reaction was stopped using 1 M H₂SO₄ (Sigma). The plates were read in Multiscan GO (Thermo Scientific) at 450 nm.

### Plaque Reduction Neutralization Test (PRNT) and Viral Load Test

Vero E6 cells were cultured in Dulbecco's Modified Eagle's Medium (DMEM) supplemented with 1% penicillin/streptomycin and 10% fetal bovine serum in 48-well plates. Mouse and hamster serum samples were inactivated at 56° C. and serially diluted in DMEM [1:10 to 1:320 (v/v)], mixed with 100 PFU of each SARS-COV2 viral stock and incubated at 37° C. to determine the viral neutralizing capacity of serum antibodies from immunized animals.

To perform the viral load assay, the right lung lobe was macerated in PBS (mice: 200 mg tissue/mL, hamster: 300 mg tissue/mL) using TissueLyser LT (Qiagen). Subsequently, the macerate was serially diluted in DMEM (undiluted to 1:100,000). For both techniques, the supernatant of Vero E6 cells was removed, and the cells were inoculated with 50 μl/well of the serum-virus mixture and incubated for 1 h at room temperature under gentle agitation, allowing infection of the cells by non-neutralized viral particles. Then, pre-warmed DMEM supplemented with 2% FBS and 2% carboxymethyl cellulose (CMC) was gently added to the plates and incubated for 4 days at 37° C. and 5% CO₂ to allow viral plaque formation. The cells were fixed with 4% formaldehyde for 2 h and stained with 1% Naphthol blue black solution (Sigma) for 1 h to visualize the plaques. The neutralizing activity was determined by the reduction in the number of plaques when compared to the positive control. The viral load was determined by counting the plaques formed.

### Measurement of IFNγ by ELISA and Flow Cytometry

Thirty days after the administration of the second dose, the animals were euthanized and splenocytes were isolated by maceration of the spleen using a 100 μm pore filter (Cell Strainer, BD Falcon). Then, the erythrocytes were lysed with ammonium chloride-potassium buffer (ACK) and the cell number was adjusted to 1×10⁶ cells/well and stimulated with 10 μg/mL of WS protein. As a positive control, Concanavalin A at 5 μg/mL (Sigma) was used. The samples were then incubated for 72 hours at 37° C. in 5% CO₂. The culture supernatant was collected for determination of IFNγ levels by ELISA (mice: R&D Systems, hamster: Mabtech) following the manufacturer's instructions.

Splenocytes: immunized mice or challenged mice were isolated as described above and plated at 2×10⁶ cells/well for intracellular IFNγ labeling. Splenocytes from immunized mice were stimulated with 10 μg/mL WS protein for 24 h and in the last 4 h PMA (50 ng/ml), Ionomycin (500 ng/UL), Golgi Plug (BD Bioscience) and Stop Golgi (BD Biosciences) were added.

Splenocytes from challenged mice were stimulated with PMA and Ionomycin alone for 4 h in the presence of Golgi Plug and Stop Golgi. Cells were stained with Live/Dead (Acqua, Invitrogen) for 20 min at 4° C. in the dark, incubated with FcBlock (BD Bioscience) for 20 min at 4° C. and then labelled with anti-CD3 (eFluor405; Cat. #48-0032-82; 1:100; eBioscience), anti-CD4 (Fitc; Cat. #553729; 1:250; BD Bioscience), anti-CD8 (APC-Cy7; Cat. #100714; 1:200; Biolenda). Cells were permeabilized with Cytofix/Cytoperm (BD Bioscience) for 20 min at 4° C. in the dark and incubated with anti-IFNγ (PerCP-Cy5.5; Cat. #45-7311-82; 1:80; eBioscience) for 30 min at 4° C. Representative density plots are presented in Supplementary FIG. 5 and the reference antibodies used in Supplementary Table 2. Flow cytometry acquisition was performed using a BD LSRFortessa and ˜200,000 live cells were acquired. Data were analyzed using FlowJo software v10.5.3.

### Flow Cytometry of Lung Tissue Cells

The mice lungs were perfused with ice-cold PBS, collected, digested with 100 μg/mL liberase (Roche), and incubated at 37° C. for 30 min for cell dissociation. The cells were filtered using a 100 μm pore cell strainer (Cell Strainer, Percoll gradient. BD Falcon) and purified using a 30% Erythrocytes were lysed with ammonium chloride-potassium (ACK) buffer and the cell number was adjusted to 2×10⁶ cells/well. The cells were stained with Live/Dead (Acqua, Invitrogen) for 20 min at 4° C. in the dark, incubated with FcBlock (BD Bioscience) for 20 min at 4° C. and then labelled with anti-CD3 (eFluor405; Cat. #48-0032-82; 1:100; eBioscience), anti-CD4 (APC; Cat. #100516; 1:200; Biolegend), anti-CD8 (APC-Cy7; Cat. #100714; 1:200; Biolegend), anti-CD19 (BV570; Cat. #115535; 1:160; Biolegend), anti-CD11b (PE-Cy7; Cat. #25-0112-82; 1:4000; eBioscience), CD11c (AF700; Cat. #56-0114-82; 1:100; eBioscience), DC-Sign (eFluor660; Cat. #50-2094-82; 1:800; eBioscience), F4/80 (PE-Cy5; Cat. #15-4801-82; 1:400; eBioscience), Ly6C (eFluor405; Cat. #48-5932-82; 1:2000; eBioscience), Ly6G (FITC; Cat. #11-9668-80; 1:250; eBioscience) and MHC II (PE; Cat. #12-5320-82; 1:400; BD Bioscience). Flow cytometry acquisition was performed using a BD LSRFortessa and ˜200,000 live cells were acquired. Data were analyzed using FlowJo software v10.5.3.

### Measurements of Cytokines and Chemokines and Viral Load by RT-PCR

For RNA isolation, a fragment of the left lung lobe was resuspended in Trizol (Invitrogen) and stored at −80° C. until RNA extraction, following the manufacturer's instructions. After total RNA extraction, samples were treated with DNase (Promega) and converted to cDNA using the High-Capacity CDNA Reverse Transcription Kit (Applied Biosystems) according to the manufacturer's instructions. For cytokine and chemokine measurements, Sybr Green PCR Master Mix (Applied Biosystems) was used to perform real-time PCR reactions on a QuantStudio 12 K Flex (Thermo Fisher Scientific) under standard conditions. qRT-PCR data were presented as 2-ΔCt. Primer sequences are presented in Table 1. qRT-PCR was performed using the GoTaq Probe RT-qPCR system (Promega) for viral load quantification, according to the manufacturer's instructions. Standard curves using serial dilutions of a plasmid containing the SARS-COV-2 gene sequence were used for sample quantification. Reactions were performed on the QuantiStudio 5 Real-Time PCR system. Probe and primer sequences used are presented in Table 1.

TABLE 1

| | SEQ | |
|---|---|---|
| | ID | |
| Gene | NO | Primer sequences |
| B-actin | 8 | F 5′ CGATGCCCTGÅGGCTCTTT 3′ |
| | 9 | R 5′ TGGATGCCACAGGATTCCAT 3′ |
| CCL2 | 10 | F 5′ TGGCTCAGCCAGATGCAGT 3′ |
| | 11 | R 5′ TTGGGATCATCTTGCTGGTG 3′ |
| CCL5 | 12 | F 5′ CAAGTGCTCCAATCTTGCAGTC 3′ |
| | 13 | R 5′ TTCTCTGGGTTGGCACACAC 3′ |
| CXCL9 | 14 | F 5′ AATGCACGATGCTCCTGCA 3′ |
| | 15 | R 5′ AGGTCTTTGAGGGATTTGTAGTGG 3′ |
| CXCL10 | 16 | F 5′ GCCGTCATTTTCTGCCTCA 3′ |
| | 17 | R 5′ CGTCCTTGCGAGAGGGATC 3′ |
| GAPDH | 18 | F 5′ GGCAAATTCAACGGCACAGT 3′ |
| | 19 | R 5′ AGATGGTGATGGGCTTCCC 3′ |
| HPRT | 20 | F 5′ GGCAAATTCAACGGCACAGT 3′ |
| | 21 | R 5′ AGATGGTGATGGGCTTCCC 3′ |
| IFN-β | 22 | F 5′ CAGCTCCAAGAAAGGACGAAC 3′ |
| | 23 | R 5′ GGCAGTGTAACTCTTCTGCAT 3′ |
| IFN-γ | 24 | F 5′ AACGCTACACACTGCATCTTGG 3′ |
| | 25 | R 5′ GCCGTGGCAGTAACAGCC 3′ |
| IL-1ß | 26 | F 5′ ACCTGTCCTGTGTAATGAAAGACG 3′ |
| | 27 | R 5′ TGGGTATTGCTTGGGATCCA 3′ |
| IL-5 | 28 | F 5′ AAAGAGAAGTGTGGCGAGGAGA 3′ |
| | 29 | R 5′ CACCAAGGAACTCTTGCAGGTAA 3′ |
| IL-6 | 30 | F 5′ TGTTCTCTGGGAAATCGTGGA 3′ |
| | 31 | R 5′ AAGTGCATCATCGTTGTTCATACA 3′ |
| IL-12 | 32 | F 5′ TGGTTTGCCATCGTTTTGCTG 3′ |
| p40 | 33 | R 5′ ACAGGTGAGGTTCACTGTTTCT 3′ |
| TNF-α | 34 | F 5′ CCCTCACACTCAGATCATCTTCT 3′ |
| | 35 | R 5′ GCTACGACGTGGGCTACAG 3′ |
| GENE E | 36 | F 5′ ACAGGTACGTTAATAGTTAATAGCGT 3′ |
| | 37 | R 5′ ATATTGCAGCAGTACGCACACA 3′ |
| | 38 | Probe FAM- |
| | | ACACTAGCCATCCTTACTGCGCTTCG-BBQ |

### Histopathology and Immunohistochemical Staining (IHC)

For histopathological analysis, lung fragments were fixed in 10% formalin for seven days after collection. The samples were processed in alcohol and xylene using the PT05 TS tissue processor (LUPETEC, UK) and embedded in histological paraffin (Histosec®, Sigma). Tissue sections of 4 μm thickness were cut using the RM2125 RTS microtome (Leica) and stained with hematoxylin and eosin.

Prior to starting the IHC staining protocol, paraffin-embedded section tissue was deparaffinized and heat-mediated antigen retrieval with Tris/EDTA buffer pH=9.0 was performed for 20 min at 60° C. Endogenous peroxidase activity was blocked with 3% hydrogen peroxide for 30 min. Slides were incubated with primary antibodies mouse anti-CD4 (Cat. #ab183685; 1:50; Abcam), mouse anti-CD8 (Cat. #ab217344; 1:200; Abcam) and mouse anti-CD19 (Cat. #ab245235; 1:200; Abcam) overnight at 4° C. The slides were then incubated with MACH 1 universal HRP polymer (Biocare Medical, USA) for 30 min at room temperature, according to the manufacturer's recommendations. Finally, the slides were stained with the chromogen 3,3′-diaminobenzidine (DAB) (Biocare Medical, USA) and counterstained with Mayer's hematoxylin. Histopathological and immunohistochemical analyses were performed by two independent pathologists.

### Statistical Analysis

The statistical analysis was performed using Prism 7.0 software for Windows (GraphPad Inc, USA). To remove possible outliers, the Grubbs test was applied, and the data distribution was verified by the Kolmogorov-Smirnoff test. The comparison between the non-immunized and immunized groups was performed using the unpaired t-test or Mann-Whitney U test, according to the data distribution.

Multiple comparison analyses were performed using ANOVA followed by the Bonferroni post-hoc test. The log-rank test was used for survival analysis. Statistical differences were considered significant when p values≤0.05.

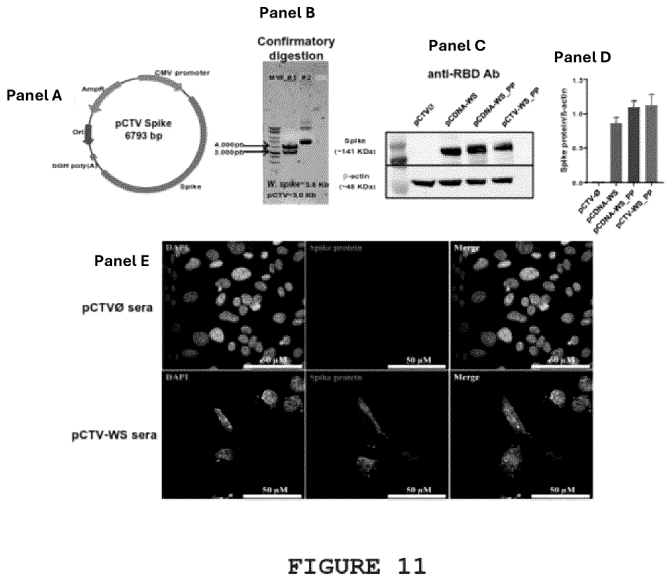

FIG. 11 shows the generation of the plasmid pCTV-WSpike (pCTV-WS). Panel a Schematic representation of the pCTV-WS plasmid used in immunogenicity and protection assays against COVID-19. Panel b Confirmatory digestion showing the presence of a ˜3.8 Kb fragment corresponding to the genetic sequence of the SARS-COV-2 S protein and the pCTV vector with an approximate size of ˜3.0 Kb. Panels c, d Representative Western blot showing WS protein expression in HEK-293 cells after transfection with pCTVØ (black bar), pCDNA-WS (green bar), pCDNA-WS PP (blue bar) and pCTV-WS PP (red bar). Panel e: Proline mutation. Immunofluorescence performed with Vero E6 cells transfected with pCTV-WS and incubated with sera from mice immunized with pCTVØ (upper panel) or pCTV-WS (lower panel). Pooled data from two independent experiments. Statistical analysis was performed using one-way ANOVA followed by Bonferroni's post-hoc test. Bars represent mean±SE.

### Construction of the DNA Vaccine Against SARS-COV-2

The codon-optimized DNA sequence encoding the complete SARS-COV-2 Spike protein(S) gene from the Wuhan-Hu-1 (WS) isolate (GenBank accession number: MN908947) was synthesized and cloned into the pCTV vector, designated pCTV-WS (FIG. 11—panel a). The WS sequence also contains a mutation (_PP) at the S1/S2 cleavage site that confers stabilization in the prefusion conformation. Restriction enzyme digestion analysis demonstrated the presence of the 3,813-bp fragment of the WS gene in pCTV-WS (FIG. 11—panel b). To compare the expression of WS protein using different plasmids, HEK293 cells were transfected with the empty vector (pCTVØ), a commercial plasmid containing the complete sequence of the WS gene of the original SARS-COV-2 isolated in Wuhan, China (pcDNA-WS), a commercial plasmid containing the codon optimized and mutated sequence of the complete S gene of SARS-COV-2 (pcDNA-WS PP) and a plasmid derived from pCDNA3.1 containing the complete codon-optimized and mutated SARS-COV-2 S gene. Neither codon optimization nor mutation altered the expression of the S protein by HEK293 cells. Furthermore, the pCTV vector showed similar expression levels when compared to pCDNA (FIG. 11—panels c, d). Vero E6 cells were transfected with pCTV-WS and immunofluorescence was performed using serum samples from mice immunized with pCTVØ or pCTV-WS. The expression of protein S by transfected T cells was observed only when serum from mice immunized with pCTV-WS was used (FIG. 11—panel e).

Immunization with pCTV-WS Induces Strong Humoral Response

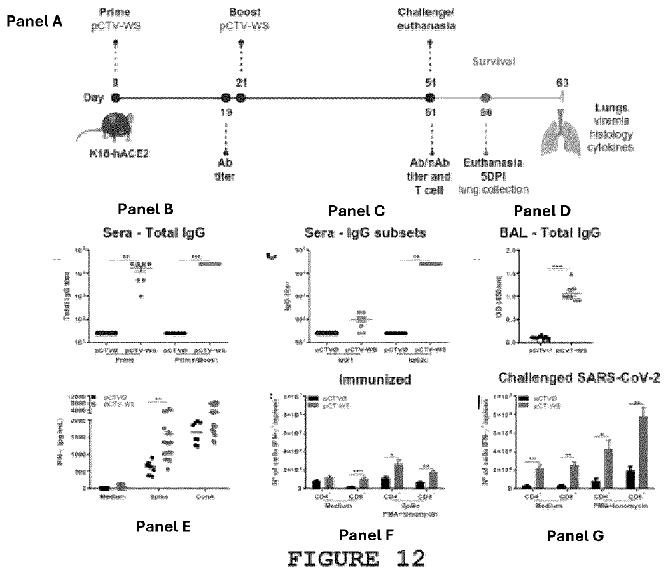

K18-hACE2 transgenic mice were immunized i.m. with two doses containing 100 μg of pCTV (Ø or WS) three weeks apart. Antibody titers were measured before the second dose and 30 days later (FIG. 12—panel a). T cell responses were also assessed 30 days after the second dose. Four/five animals from each group were challenged with SARS-COV-2 and followed for 12 days for survival assessment or euthanized 5 days post infection (DPI) for determination of viral load, inflammatory profile, and histopathological analysis (FIG. 12—panel a). After the first dose of pCTV-WS (prime), mice developed significant levels of total anti-WS IgG (FIG. 12b-gray circles). These levels increased 30 days after the second dose (booster) (FIG. 12—panel b-red circles). When the levels of IgG isotypes were determined, we observed that IgG2c was predominant in mice immunized with pCTV-WS (FIG. 12—panel c). Significant levels of total anti-WS IgG were also found in the bronchoalveolar lavage fluid (BALF) (p<0.001).

In addition to the strong humoral immune response, a significant cellular response was also observed. Splenocytes from animals immunized with pCTV-WS produced IFN-γ in response to restimulation with WS protein compared to the control group (p<0.01) (FIG. 12—panel e). Mice immunized and then challenged with SARS-COV-2 showed a higher number of IFN-γ-producing CD4+ and CD8+ T cells compared to CD4+ and CD8+ T cells from immunized mice restimulated in vitro with S protein from the Wuhan isolate (FIG. 12—panels f, g).

FIG. 12 shows the immunogenicity and protection of pCTV-WS in K18-hACE2 immunized mice. Panel a show the schematic representation of the experimental design used to evaluate the humoral and cellular immune response and protection against SARS-COV-2. Panel b shows the total anti-WS IgG titer in mice immunized with pCTVØ (open circles) or pCTV-WS (gray circles) after 1st immunization (prime) and pCTVØ (filled circles) or pCTV-WS (red circles) after priming/boosting [n=8 mice/group]. Panel c IgG1 (blue circles) and IgG2c (red circles) subclass 30 days after pCTV-WS priming/boosting immunization. IgG1 (open circle) and IgG2c (filled circle) in the control group [n=8 mice/group]. Panel d Total anti-WS IgG in bronchoalveolar lavage (BALF) of mice immunized with pCTVØ (black circles) or pCTV-WS (red circles) [n=8 mice/group]. Panel e Levels of IFN-γ produced by splenocytes from mice immunized with pCTVØ (black circles) or pCTV-WS (red circles) after 72 h of incubation with medium (negative control), restimulation with recombinant WS protein or Concanavalin A (positive control) [n=8, pCTVØ and n=20, pCTV-WS]. IFN-γ production by CD4+ T cells and CD8+ T cells from immunized mice stimulated in vitro with WS protein panel f or of mice challenged with Wuhan strain at 5 DPI panel g (n=4 for both groups). Pooled data from two independent experiments. Statistical analysis of IgG and IFN-γ levels was performed using the unpaired t-test or Mann-Whitney U test, according to the data distribution. Bars represent mean±SEM. * p<0.05; ** p<0.01; *** p<0.001.

Immunization with pCTV-WS Protects Against the Wuhan Strain and Reduces Viral Load

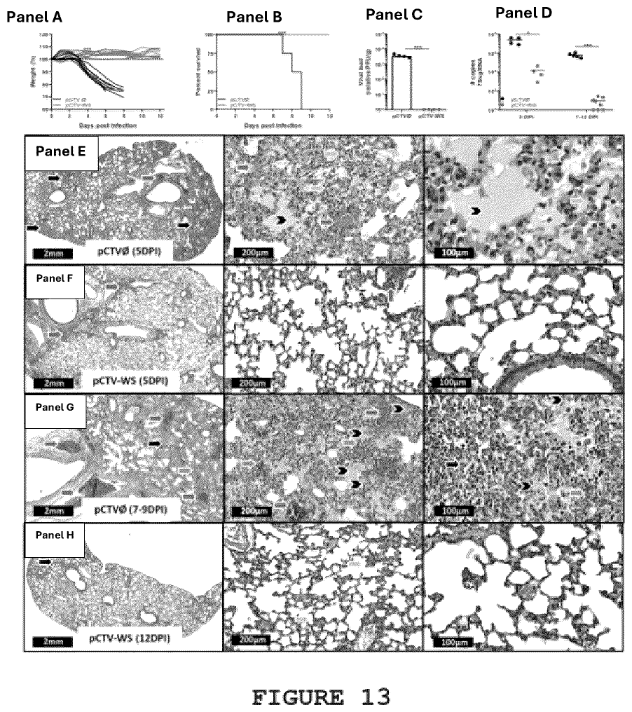

All animals immunized with pCTV-WS and challenged with SARS-COV-2 maintained their body weight and survived the challenge. On the other hand, all animals in the control group lost weight from the 4 DPI onwards and died by the 8 DPI (p<0.001) (FIG. 13—panel a, panel b). The viral load was measured in lung samples at 5 DPI. Immunized animals showed no PFUs while the control group exhibited infection rates higher than 10³ PFUS/g tissue (FIG. 13—panel c). Despite the absence of viable virus in immunized animals, lower levels of viral RNA were found in lung samples at 5 DPI (p<0.05) and 7-12 DPI (p<0.001) compared to the control group (FIG. 13—panel d). Histopathological analysis of lung samples from non-immunized mice (pCTVØ) euthanized on the 5 DPI showed loss of alveolar architecture with intense and diffuse inflammatory infiltrate (black arrow), thickening of the alveolar septum (yellow arrow), edema (arrowhead) and vascular congestion (blue arrow) (FIG. 13—panel e). On the other hand, the animals immunized with pCTV-WS only presented perivascular inflammatory infiltrate (red arrow) with preserved lung parenchyma (FIG. 13—panel f). At the 7-9 DPI the pCTVØ mice exhibited an intense and diffuse inflammatory infiltrate (black arrow) associated with vascular congestion (blue arrow), bleeding (green arrow) and edema (black arrowhead) (FIG. 13—panel g). Mice immunized with pCTV-WS at 12 DPI presented preserved alveolar areas with some inflammatory focus (black arrow) and thickening of the alveolar septum (yellow arrow) (FIG. 13—panel h).

FIG. 13 shows protection and viral load of K18-hACE2 mice immunized with pCTV-WS. Body weight (panel a) and survival (panel b) of mice immunized with pCTVØ (black lines) or pCTV-WS (red lines) and challenged with the Wuhan strain of SARS-COV-2 [n=8 mice/group]. Panel c—Lung viral load in K18-hACE2 mice immunized with pCTVØ (black bar) or pCTV-WS (red bar) and challenged with the Wuhan strain at 5 DPI [n=4 mice/group]. Panel d-Number of RNA copies of SARS-COV-2 in K18-hACE2 mice immunized with pCTVØ (black circles) or pCTV-WS (red circles) and challenged with the Wuhan strain at 5 and 7-12 DPI [n=4-5, pCTVØ and n=4-8 pCTV-WS]. Histopathological sections of lungs of K18-hACE2 mice immunized with pCTVØ panel e, panel g and pCTV-WS panel f, panel h and at 5 DPI and 7-12 DPI, respectively [n=4 mice/group]. Inflammatory infiltrate (black arrow), Perivascular inflammatory infiltrate (red arrow), thickening of the alveolar septum (yellow arrow), vascular congestion (blue arrow), edema (arrowhead) and bleeding (green arrow) at 1.1×, 10× and 20× magnification. The statistical analysis of the weight curves was performed by area under the curve followed by unpaired t-test. The statistical analysis of survival was performed using the log-rank test. The statistical analysis of viral load was performed using the unpaired t-test. * p<0.05; *** p<0.001.

Immunization with pCTV-WS Reduces Inflammation and Induces Recruitment of B and T Cells to the Lungs

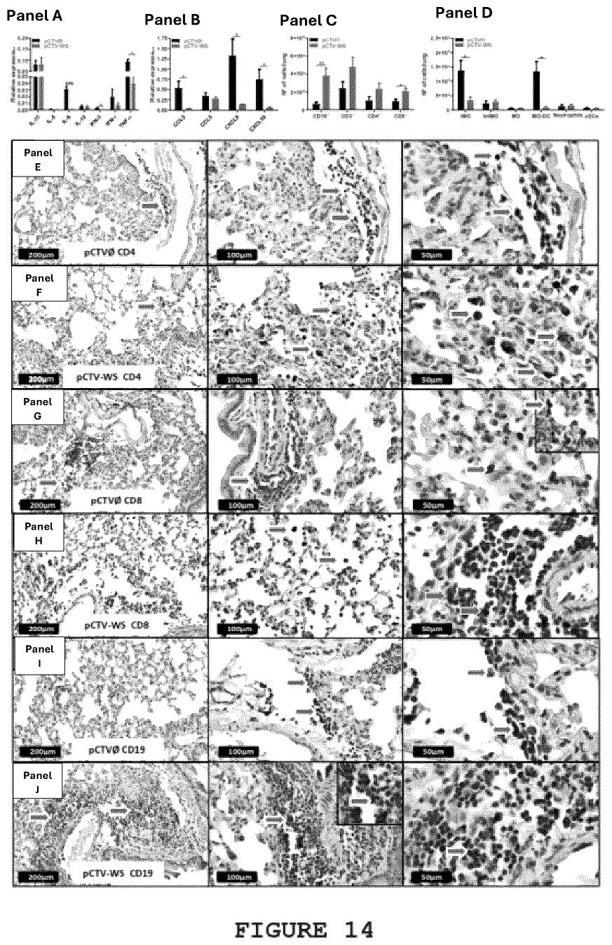

A pronounced inflammatory reaction was observed in lung samples from the control group, as indicated by the presence of inflammatory cytokines such as IL-6, IFN-β and TNFα, as well as chemokines such as CCL2, CXCL9 and CXCL10 at higher levels in the control group compared to the immunized animals (FIG. 14—panel a, panel b). The animals immunized with pCTV-WS showed an increase in the number of B cells (CD19+) and CD8+ T cells in the lungs at 5 DPI (FIG. 14—panel c). In contrast, the control group exhibited a strong inflammatory reaction with inflammatory monocytes and monocyte-derived DC (MO-DC) (FIG. 14—panel d). Immunohistochemical analyses showed diffuse CD4+ and CD8+ T cells in the lung tissue (FIG. 14—panels e-h) and B cells (CD19+) more restricted to the perivascular region (FIG. 14—panels i, j).

FIG. 14 shows the inflammatory profile and immunohistochemical staining of B and T cells in lung samples of K18-hACE2 mice immunized with pCTV-WS. Relative expression of cytokines (panel a) and chemokines (panel b) in lung samples from mice immunized with pCTVØ (black bar) or pCTV-WS (red bar) and challenged with the Wuhan strain at 5 DPI [n=4 mice/group]. Absolute number of B cells (CD19+), T cells (CD3+), CD4+ and CD8+ T cells (panel c) and inflammatory monocytes (F4/80+CD11b+Ly6Chigh), intermediate monocytes (F4/80+CD11b+Ly6Cint), monocytes (F4/80+CD11b+Ly6C-), monocyte-derived dendritic cells (F4/80+CD11b+Ly6ChighDC-Sign+), neutrophils (F4/80-CD11bhighLy6G+) and classical dendritic cells (CD11b−/lowCD11c+MHC-II+) (panel d) at 5 DPI in lung samples of K18-hACE2 immunized mice pCTVØ (black bar) and pCTV-WS (red bar), by flow cytometry [n=4 mice/group]. Immunohistochemical staining of lung tissue sections from mice immunized with pCTVØ and pCTV-WS and challenged with Wuhan strain, labelled with anti-CD4 (panel e, panel f, anti-CD8 panel g, panel h and anti-CD19 panel i, panel j showing the location of CD4+ T cells, CD8+ T cells and B cells (red arrows) in lung tissue at 10×, 20× and 40× magnification. The statistical analysis was performed using the unpaired t-test. Bars represent mean±SE. * p<0.05; ** p<0.01; *** p<0.001.

Immunization with pCTV-WS Protects Against SARS-COV-2 Variants of Concern in the Severe COVID-19 Experimental Model

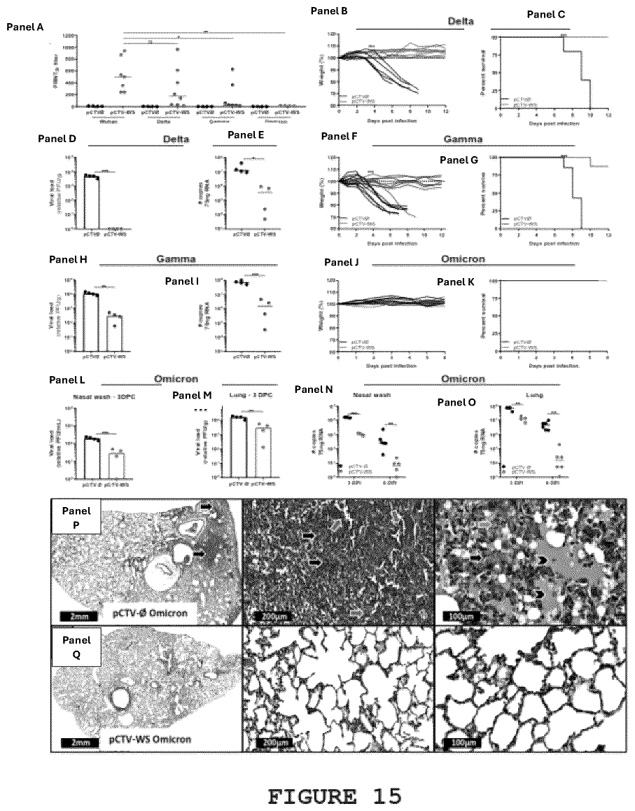

Neutralizing antibodies against different variants of SARS-COV-2 were evaluated in serum samples from mice immunized with pCTV-WS. The PRNT₅₀ titer was approximately 500 for the Wuhan strain (FIG. 15—panel a). Although the PRNT₅₀ titer was reduced for the Delta variant, this reduction was not statistically significant. A drastic reduction in neutralizing capacity was observed for the Gamma and Omicron variants, with no animals showing a neutralizing titer for Omicron (FIG. 15—panel a).

Mice immunized with pCTV-WS were also challenged with the Delta, Gamma, and Omicron variants. The pCTV-WS protected 100% of animals challenged with the Delta variant (FIG. 15—panel b, panel c). These animals did not lose weight (p<0.001) and presented 100% survival (p<0.001), compared to 100% mortality in the control group. Mice immunized and challenged with the Delta variant did not present viable viral load, while the control group exhibited more than 10³ PFU/g of tissue (FIG. 15—panel d). Despite the absence of viable virus in immunized animals, viral RNA was found in lung samples at 5 DPI (p<0.05) (FIG. 15e). For mice challenged with the Gamma variant, one immunized mouse died on the 9 DPI and the observed protection was almost 90% (FIG. 15—panel f, panel g). A significant reduction in viable viral load and viral RNA was observed in lung samples from mice immunized and challenged with the Gamma variant (p<0.01) (FIG. 15—panel h, panel i). Knowing that infection with the Omicron variant results in non-lethal infection, after challenge with Omicron, viral load was assessed in nasal wash and lung. Immunized mice were euthanized on 3 and 6 DPI and no change in weight and survival was noted (FIG. 15—panel j, panel k).

After challenge with Omicron, mice immunized with pCTV-WS showed a significant reduction in viable viral load at 3 DPI in nasal wash and lung compared to the control group (p<0.01) (FIG. 15—panel 1, panel m). The persistence of viral RNA, also assessed in nasal wash and in the lung at 3 and 6 DPI, showed a significant reduction in animals immunized with pCTV-WS at both time points, compared to the control group (p<0.01) (FIG. 15—panel n, panel o). Histopathological analysis of non-immunized animals challenged with the Omicron variant showed intense and diffuse inflammatory infiltrate (black arrows)) throughout the lung parenchyma associated with perivascular inflammatory infiltrate (red arrow), congestion (blue arrow) and bleeding (green arrow) (FIG. 15—panel p). On the other hand, mice immunized with pCTV-WS presented preserved lung parenchyma and intact alveolar spaces (FIG. 15—panel q).

FIG. 15 shows protection against variants of concern of SARS-COV-2 in K18-hACE2 mice immunized with pCTV-WS. Panel a—PRNT₅₀ was evaluated using different SARS-COV-2: Wuhan strain (lineage B), Delta, Gamma, and Omicron variants in serum samples of K18-hACE2 mice immunized with pCTVØ (black circles) or pCTV-WS (colored circles) [n=4, pCTVØ and n=4-8, pCTV-WS]. Body weight (panel b) and survival (panel c) of K18-hACE2 mice immunized with pCTVØ (black lines) or pCTV-WS challenged with Delta (green lines). Viable viral load (panel d) and number of RNA copies of SARS-COV-2 and at 5 DPI in mice immunized with pCTVØ (black circles) and pCTV-WS (green circles) and challenged with the Delta variant. [n=4-6, pCTVØ and n=4-8 pCTV-WS]. Body weight (panel f) and survival (panel g) of K18-hACE2 mice immunized with pCTVØ (black lines) or pCTV-WS and challenged with the Gamma variant (blue lines). Viable viral load (panel h) and number of RNA copies of SARS-COV-2 (panel i) at 5 DPI in mice immunized with pCTVØ (black circles) and pCTV-WS (blue circles) and challenged with the Gamma variant [n=4−6, pCTVØ and n=4-8 pCTV-WS]. Body weight (panel j) and survival (panel k) of K18-hACE2 mice immunized with pCTVØ (black lines) or pCTV-WS challenged with the Omicron variant (pink line). Viral load at nasal wash (panel 1) and lung (panel m) of K18-hACE2 immunized mice pCTVØ (black bar) or pCTV-WS (pink bar) and challenged with the Omicron variant at 3 DPI [n=4 mice/group]. Number of RNA copies do SARS-COV-2 in nasal wash (panel n) and lung (panel o) of K18-hACE2 immunized mice pCTVØ (black circles) or pCTV-WS (pink circles) and challenged with the Omicron variant at 3 and 6 DPI [n=4-6 mice/group]. Histopathological sections of lungs of K18-hACE2 immunized mice pCTVØ (panel p) or pCTV-WS (panel q) and challenged with the variant Omicron at 6 DPI. Inflammatory infiltrate (black arrow), perivascular inflammatory infiltrate (red arrow), vascular congestion (blue arrow), edema (arrowhead) and bleeding (green arrow) at 1.1×, 10× and 20× magnification. Pooled data from one or two independent experiments. The statistical analysis of the weight curves was performed by area under the curve followed by unpaired t-test or Mann-Whitney test, according to data distribution. The statistical analysis of the survival was performed using the log-rank test. The statistical analysis of the viral load and the number of RNA copies was performed using the unpaired t-test or Mann-Whitney test, according to data distribution. * p<0.05; ** p<0.01; *** p<0.001.

Immunization with pCTV-WS Also Induces a Strong Humoral Immune Response and Protective Immunity in Hamsters

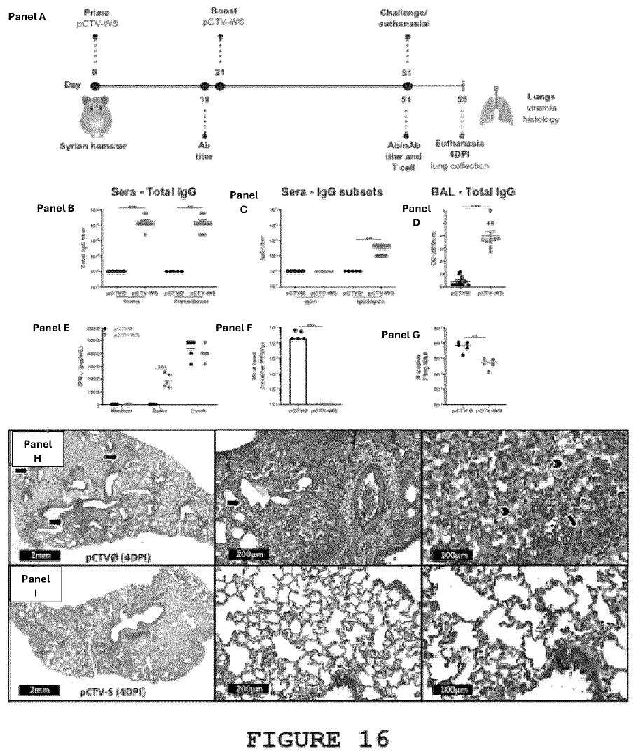

Hamsters were immunized with two i.m. doses of 100 μg of plasmid 21 days apart in order to assess the immunogenicity of the vaccine candidate (FIG. 16—panel a). pCTV-WS induced high levels of serum IgG in hamsters both after the priming dose and after the boost (FIG. 16—panel b), and in the BALF after the priming/boosting (p<0.001) (FIG. 16—panel d). IgG subclass analysis showed that immunization elicited both IgG2 and IgG3 against the WS protein (FIG. 16—panel c). Splenocytes isolated from hamsters 30 days after the second dose of pCTV-WS and restimulated with 10 μg/mL of recombinant WS protein secreted high levels of IFN-γ (p<0.001) (FIG. 16—panel e).

Although hamsters immunized with pCTV-WS had a viral load below the limit of detection (FIG. 16—panel f), viral RNA persisted in lung samples at 4 DPI at 10-fold lower levels compared to control animals (p<0.01) (FIG. 16—panel g). Histopathological analysis of non-immunized animals showed several foci of inflammation (black arrows) with severe inflammation around vessels and bronchi (red arrow) and thickening of the alveolar septum (yellow arrow) (FIG. 16—panel h). The lungs of these animals also present hemorrhagic foci (green arrow) and edema (arrowhead). (FIG. 16—panel h). On the other hand, hamsters immunized with pCTV-WS showed much fewer inflammatory infiltrates compared to control animals and large areas of preserved lung parenchyma (FIG. 16—panel i).

FIG. 16 shows the immunogenicity and protective immunity of the pCTV-WS in the Syrian hamster model. Panel A—Schematic representation of the experimental design used to evaluate humoral and cellular immune response and protection against the SARS-COV-2. Panel B—Total anti-WS IgG titer in hamsters immunized with pCTVØ (open circles) or pCTV-WS (grey circles) after the 1st immunization (prime) and pCTVØ (filled circles) or pCTV-WS (red circles) after priming/boosting [n=5, pCTVØ and n=12, pCTV-WS]. Panel C—Subclass IgG1 (blue circles) and IgG2/IgG3 (red circles) 30 days after priming/boosting immunization with pCTV-WS IgG1 (open circles) and IgG2/IgG3 (filled circles) in the control group [n=5, pCTVØ and n=12, pCTV-WS]. Panel D-Total anti-WS IgG in bronchoalveolar lavage fluid (BALF) from hamsters immunized with pCTVØ (black circles) or pCTV-WS (red circles) [n=10 mice/group]. Panel e—IFN-γ levels secreted by hamster splenocytes immunized with PCTVØ (black circles) or pCTV-WS (red circles) after 72 h of incubation with medium (negative control), restimulation with recombinant WS protein or Concanavalin A (positive control). Pulmonary viral load (panel f) and number of RNA copies of SARS-CoV-2 (panel g) in hamsters immunized with pCTVØ (black circles) or pCTV-WS (red circles) and challenged with the Wuhan strain at 4 DPI [n=5 mice/group]. Histopathological sections of hamster lungs immunized with pCTVØ (panel h) or pCTV-WS (panel i) at 4 DPI. Inflammatory infiltrate (black arrow), Perivascular inflammatory infiltrate (red arrow), thickening of the alveolar septum (yellow arrow), edema (arrowhead) and bleeding (green arrow) at 1.1×, 10× and 20× magnification. The statistical analysis was performed using the unpaired t-test or Mann-Whitney test, according to data distribution. ** p<0.01; *** p<0.001.

Immunization with pCTV-WS also induces protection against SARSCoV-2 variants in a moderate experimental model of COVID-19

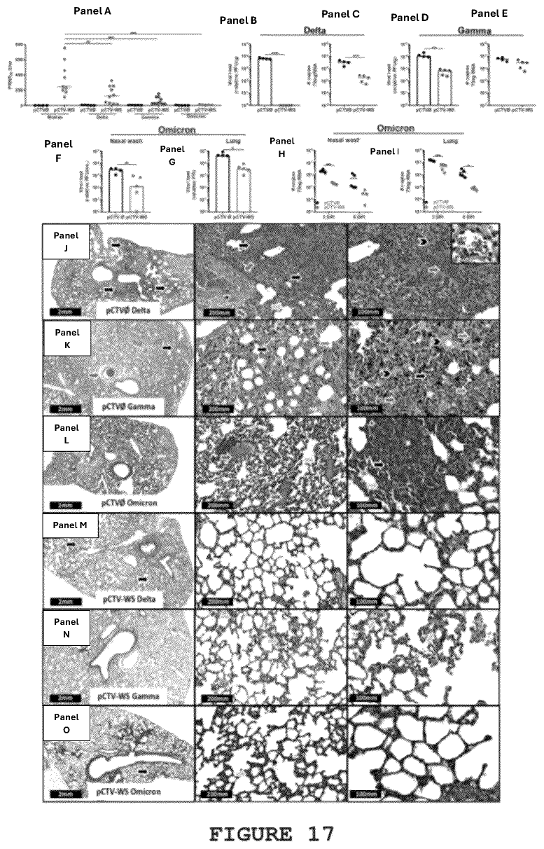

Sera of all immunized hamsters tested positive for neutralizing antibodies for Wuhan SARS-COV-2 (FIG. 7—panel a). PRNT50 titers were highest against Delta and lowest against Omicron. A significant reduction in nAb titer was observed for all variants (p<0.01), being more pronounced against the Omicron variant with no animal showing neutralization titer (FIG. 17—panel a). Viral load was also assessed in lung samples at 3-4 DPI for the three variants (FIG. 17—panels b-i). After challenge with the Delta variant, immunized animals showed no PFU (p<0.001) and lower levels of viral RNA (p<0.001) in lung samples when compared to the control group (FIG. 17—panel b, panel c). immunization with pCTV-WS was also able to reduce the number of PFUs in the lung of hamsters challenged with the Gamma variant by approximately 10 times compared to the control group (p<0.01) (FIG. 17—panel d). No statistically significant differences were observed in lung viral RNA levels at 4 DPI (FIG. 17—panel e). The animals immunized with pCTV-WS also presented reduced viral load both in nasal wash (FIG. 17—panel f) and lung (FIG. 7—panel g) at 3 DPI with the Omicron variant (p<0.05). Lower levels of viral RNA were also detected at 3 DPI in nasal wash of immunized animals (p<0.001) (FIG. 7—panel h). Reduced levels of viral RNA were detected at 3 and 6 DPI in lung tissue (p<0.05) (FIG. 17—panel i). Histopathological analyses were performed on hamster tissues that were challenged with the three different SARS-COV-2 variants. Hamsters immunized with pCTVØ and challenged with the Delta, Gamma and Omicron variants presented diffuse inflammatory tissue infiltrate (black arrow), perivascular inflammatory infiltrate (red arrow), thickening of the alveolar septum (yellow arrow), vascular congestion (blue arrow) and edema (arrowhead) (FIG. 17—panels j-l). The bleeding was only observed in animals challenged with the Gamma variant (green arrow) (FIG. 17—panel k). On the other hand, the animals immunized with pCTV-WS maintained tissue architecture and only a mild inflammatory infiltrate (black arrow), perivascular inflammatory infiltrate (red arrow) and thickening of the alveolar septum (yellow arrow) (FIG. 17—panels m-o).

FIG. 17 shows protection against variants of concern of SARS-COV-2 in Syrian hamsters immunized with pCTV-WS. Panel a—PRNT₅₀ was assessed using different SARS-COV-2: Wuhan strain (lineage B), Delta, Gamma, and Omicron variants in serum samples from cpCTVØ hamsters (black circles) or pCTV-WS (colored lines) [n=4-5, pCTVØ and n=4-10, pCTV-WS]. Viral load (panel b) and number of RNA copies of SARS-COV-2 (panel c) in lung samples of immunized pCTVØ (black circles) or pCTV-WS (green circles) hamsters and challenged with the Delta variant of SARS-COV-2 at 4 DPI [n=4-5 hamsters/group]. Viral load (panel d) and number of RNA copies of SARS-COV-2 and in lung samples from hamsters immunized with pCTVØ (black circles) or pCTV-WS (blue circles) and challenged with the Gamma variant of SARS-COV-2 at 4 DPI [n=4-5 hamsters/group]. Viral load in nasal wash (panel f) and lung (panel g) from hamsters immunized with pCTVØ (black bar) or pCTV-WS (pink bar) and challenged with the Omicron variant at 3 DPI [n=4-5 hamsters/group]. Number of RNA copies of SARS-COV-2 in the nasal wash (panel h) and lung tissue (panel i) from hamsters immunized with pCTVØ (black circles) or pCTV-WS (pink circle) and challenged with the Omicron variant at 3 and 6 DPI [n=4-5 hamsters/group]. Histopathological sections of lungs of hamsters immunized with pCTVØ (panels j-l) or pCTV-WS (panels m-o) and challenged with the Delta, Gamma, and Omicron variants at 4 DPI, respectively. Inflammatory infiltrate (black arrow), Perivascular inflammatory infiltrate (red arrow), thickening of the alveolar septum (yellow arrow), vascular congestion (blue arrow), edema (arrowhead) and bleeding (green arrow) at 1.1×, 10× and 20× magnification [n=4-5 hamsters/group]. The statistical analysis was performed using the unpaired t-test or Mann-Whitney test, according to data distribution. * p<0.05; ** p<0.01; *** p<0.001.

Differential Requirement of nAbs and T Cells in Protective Immunity Induced by pCTV-WS

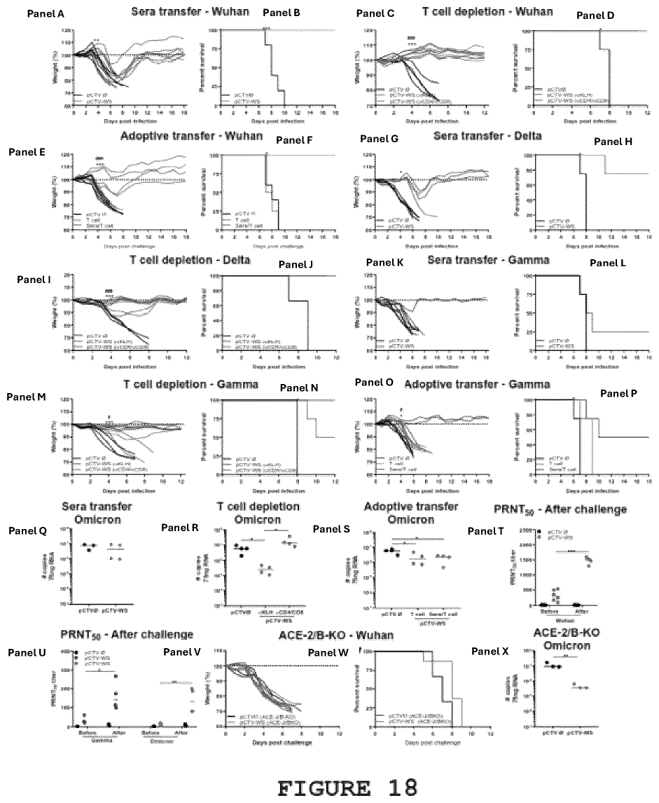

Serum samples collected from mice that received 2 doses of pCTVØ or pCTV-WS were transferred to recipient mice one day before challenge with SARS-COV-2 from Wuhan. Initially, all mice lost weight regardless of the sera transferred. However, mice that received a serum sample from a donor immunized with pCTV-WS recovered the initial body weight (p<0.01) and survived until 18 DPI, while all mice that received serum from a pCTVØ donor died until 10 DPI (p<0.001) (FIG. 18—panel a, panel b). It was also investigated whether the T cells are essential to the protective immunity against SARS-COV-2. On days −3, −2 and −1 before the challenge, K18-hACE2 mice immunized with pCTV-WS were treated with anti-CD4 and anti-CD8 antibodies or anti-KLH (isotype control) antibodies. The depletion of T cells did not influence the course of the infection, and all mice challenged with the Wuhan strain maintained the body weight (p<0.001) and survived (p<0.05) (FIG. 18—panel c, panel d). The adoptive transfer of T cells did not protect the mice from infection and all animals died by 9 DPI, indicating the importance of nAbs against the Wuhan strain (FIG. 18 e, f).

For the Delta variant, high protection was found (75% survival) in mice that received sera from mice immunized with pCTV-WS (FIG. 18—panel g, panel h). Furthermore, all mice immunized with pCTV-WS that had T cells depleted survived (FIG. 18—panel i, panel j). In contrast, only partial protection was observed (25% survival) for the Gamma variant in K18-hACE2 mice who received sera from mice immunized with pCTV-WS before the challenge (FIG. 18—panels k-l). Furthermore, after the challenge with the Gamma variant, the survival fell to 50% in mice immunized with depletion of T cells (FIG. 8—panel m, panel n). The adoptive transfer of T cells did not protect mice from infection by the Gamma variant and all mice died at 9 DPI. However, simultaneous transfer of immune sera and T cells from vaccinated mice protected 50% of the animals, demonstrating the importance of humoral and cellular immune responses against this variant (FIG. 18—panel o, panel p).

For the Omicron variant, we also analyzed the viral RNA load in mice that received serum transfer and in mice pCTV-WS immunized who have been depleted of T cells. The mice that received serum transfer presented similar viral RNA load when compared to the non-immunized control group (FIG. 18—panel q). On the other hand, animals with depletion of T cells presented higher viral RNA load compared to immunized mice that received the isotype control (αKLH) and a similar load to non-immunized animals (pCTVØ) (FIG. 18—panel r). The adoptive transfer of T cells with or without serum transfer (p<0.05) showed a similar reduction in viral RNA load in K18-hACE2 mice (FIG. 18—panel s), suggesting the absence of nAbs against this variant. Furthermore, PRNT₅₀ was calculated before and after the challenge. The sera from immunized mice and challenged with the Wuhan strain showed a significant increase in neutralizing capacity (p<0.001) (FIG. 18—panel t).

However, immunized mice challenged with the Gamma and Omicron variants showed 10 times lower neutralizing capacity (p<0.05) compared to mice challenged with Wuhan (FIG. 18—panel u), further questioning the efficacy of nAbs in protective immunity against the Gamma and Omicron variants.

The inventors of the present application further analyzed the body weight and the survival of K18-hACE2/B-KO mice immunized with pCTV-WS and challenged with the Wuhan strain or the Omicron variant. All mice died at 9 DPI, confirming the importance of nAbs in protecting against the ancestral strain (FIG. 18—panels v-w). On the other hand, K18-hACE2/B-KO mice immunized with pCTV-WS and challenged with the Omicron variant showed a reduction in the viral RNA load (p<0.01), similar to vaccinated K18-hACE2 mice (FIG. 18—panel x). Together, those results show that a protective immunity against the Wuhan strain (lineage B) and Delta variant is measured primarily by nAbs, whereas protection of mice immunized against Gamma appears to be mediated both by nAbs and T cells. It is important to highlight that the protective immunity for the Omicron variant is predominantly mediated by T cells.

FIG. 18 shows neutralizing antibodies and protective immunity in K18-hACE2 mice immunized with pCTV-WS. Body weight (panel a) and survival (panel b) of K18-hACE2 mice after serum transfer from immunized pCTVØ (black lines) or pCTV-WS (red lines) mice followed by challenge with the Wuhan strain [n=5, pCTVØ and n=7 pCTV-WS]. Body weight (panel c) and survival (panel d) of K18-hACE2 mice immunized with pCTVØ (black lines) and pCTV-WS, treated with αKLH (red lines) or αCD4/αCD8 (purple lines) and challenged with the Wuhan strain [n=4 mice/group]. Statistical analysis: [*pCTVØ vs pCTV-WS (αKLH)] [#pCTVØ VS pCTV-WS (αCD4/αCD8)]. Body weight (panel e) and survival (panel f) of K18-hACE2 mice (black lines), which received adoptive transfer of T cells (purple lines) or adoptive transfer of T cells+serum transfer (red lines) followed by challenge with the Wuhan strain [n=4 mice/group]. Statistical analysis: [*pCTVØ VS sera+ T cells] [#sera+ T cells vs T cells]. Body weight (panel g) and survival (panel h) of K18-hACE2 mice (black lines) which received serum transfer (green lines) followed by challenge with the Delta variant [n=4 mice/group]. Body weight (panel i) and survival (panel j) of K18-hACE2 mice immunized with pCTVØ (black lines) and pCTV-WS, treated with αKLH (green lines) or αCD4/αCD8 (purple lines) and challenged with the Delta variant [n=3-4 mice/group]. Statistical analysis: [*pCTVØ vs pCTV-WS (αKLH)] [#pCTVØ VS pCTV-WS (αCD4/αCD8)]. Body weight (panel k) and survival (panel 1) of K18-hACE2 mice (black lines) which received serum transfer (blue lines) followed by challenge with the Gamma variant [n=4 mice/group]. Body weight (panel m) and survival (panel n) of K18-hACE2 mice immunized with pCTVØ (black lines) and pCTV-WS, treated with αKLH (blue lines) or αCD4/αCD8 (purple lines) and challenged with the Gamma variant [n=3-4 mice/group]. Statistical analysis: [*pCTVØ vs pCTV-WS (αKLH)] [#pCTVØ vs pCTV-WS (αCD4/αCD8)]. Body weight (panel o) and survival (panel p) of K18-hACE2 mice (black lines), which received adoptive transfer of T cells (purple lines) or adoptive transfer of T cells+serum transfer (blue lines) followed by challenge with the Gamma variant [n=4 mice/group]. Statistical analysis: [*pCTVØ vs sera+ T cells] [#pCTVØ vs T cells]. (panel q) SARS-CoV-2 RNA copy number of K18-hACE2 mice who received serum transfer from mice immunized with pCTVØ (black circles) or pCTV-WS (pink circles) and challenged with the Omicron variant at 6 DPI [n=4 mice/group]. (panel r) SARS-COV-2 RNA copy number of K18-hACE2 mice immunized with pCTVØ (black circles) and pCTV-WS who received isotype control antibody (αKLH-pink circles) or αCD4/αCD8 (purple circles) and challenged with the Omicron variant at 6 DPI [n=4 mice/group]. (panel s) Viral RNA load of K18-hACE2 mice who received adoptive transfer of T cells (purple circles) or adoptive transfer of T cells+serum transfer (pink circles) and who were challenged with the Omicron variant at 6 DPI [n=4 mice/group. PRNT50 of K18-hACE2 mice immunized with pCTVØ (black circles) or pCTV-WS (colored circles) before and after challenge with the Wuhan strain (panel t) or with Gamma and Omicron variants (panel u) [n=4-6 mice/group]. Body weight (panel v) and survival (panel w) of K18-hACE2/B-KO mice immunized with pCTVØ (black lines) or pCTV-WS (red lines) and challenged with the Wuhan strain [n=3-9 mice/group] (panel x) Viral RNA load of K18-hACE2/B-KO mice immunized with pCTVØ (black circles) or pCTV-WS (pink circles) and challenged with the Omicron variant [n=3 mice/group]. Pooled data from one or two independent experiments. The statistical analysis of the weight curves was performed using the area under the curve followed by the unpaired t-test or Mann-Whitney test, according to the data distribution. The statistical analysis of the survival was performed using the log-rank test. The statistical analysis for RNA copies number was performed by using unpaired t-test or Mann-Whitney test, according to the data distribution. *p<0.05; ** p<0.01; *** p<0.001.

Although vaccine coverage against SARS-COV-2 is widespread in most high- and middle-income countries, the COVID-19 pandemic continues with new waves of high transmission. The escape of emerging variants and the waning of vaccine-induced immunity contribute to the limited control of the COVID-19 pandemic.

However, it seems that vaccination will continue to be essential to reduce severe cases and deaths. We have developed a plasmid DNA vaccine containing the complete Spike protein gene of the Wuhan strain (pCTV-WS), which may be useful as a booster vaccine in the context of new variants. Pre-clinical studies using animal models have provided important data on pathogenicity, transmissibility, as well as escape of VOCs from immunity induced by infection or vaccination. This information can be used to establish the level of protection of vaccines produced against the ancestral strain and the immunological mechanisms involved. Non-human primates (NHP) have been widely used to test COVID-19 vaccines against SARS-COV-2 variants. Although NHPs are good models for SARS-COV-2 infection, they are not good models for the severe form of COVID-19. Here, we evaluated the efficacy of pCTV-WS in K18-hACE2 mice and hamsters, which are models of severe and moderate COVID-19, respectively. The results showed that, although pCTV-WS-induced nAbs are critical for controlling infection with the ancestral Wuhan virus and the Delta variant, T cells played a critical role in controlling viral load and disease in mice immunized and infected with the Gamma or Omicron VOCs.

The Omicron variant has spread rapidly globally and is the variant with the most notable antibody escape to date. Lower in vitro neutralization titers of sera from convalescent and vaccinated individuals are consistent with the reduced efficacy of vaccines in use against Omicron and other variants. Although the variant can infect and cause disease in vaccinated or convalescent individuals, the innate immunity, the response of pre-existing T cells, the non-neutralizing antibodies and the residual nAbs seem to protect against severe disease, reducing hospitalizations and deaths. Although booster doses improve immunity, some studies also indicate that booster doses with vaccines produced on different platforms induce more robust immune responses against variants, albeit for short periods of time.

To date, four vaccines based on DNA encoding the S protein against SARS-COV-2 are underway with phase 3 clinical trials. Preliminary data reported from three of these vaccines have shown that they are effective, safe, well tolerated and induce humoral and cellular immune responses. The inventors of the present application herein showed that two doses of pCTV-WS (SEQ ID NO. 4) three weeks apart induced high levels of nAbs and robust T cell responses in both mice and hamsters. Histopathological analysis of animals challenged with Wuhan's strain and different variants showed that vaccinated mice and hamsters had preserved lung structure, reduced inflammatory infiltrate, levels of pro-inflammatory cytokines and chemokines compatible with controlled viral replication, compared to unvaccinated animals. Neutralization assays with sera from mice or hamsters immunized with pCTV-WS (SEQ ID NO. 4) showed cross-neutralization to the ancestral B lineage of SARS-COV-2 and the Delta variant. However, it failed to induce high levels of nAbs to the highly virulent Gamma isolate. Consistent with the multiple mutations in the S protein, the Omicron variant completely escaped the nAbs induced by DNA-WS, in vitro and also in vivo.

Although a decrease in the antibodies was observed in those vaccinated with mRNA, memory T cells and B cells remained relatively stable. Although T cells cannot prevent infection, the immunity conferred by these cells was still essential to limit virus replication and spread to the lower respiratory tract and prevent severe disease. Another study showed that T cell responses are largely preserved to Omicron Spike and non-Spike proteins in ˜80% of previously infected and/or vaccinated individuals. The booster vaccination also increased the reactivity of T cells to Omicron's Spike. Thus, it is possible that protection against severe disease for 6 months among vaccinated individuals is mediated by T cells. It is important to highlight that vaccines based on mRNA, adenovirus and recombinant proteins induce strong responses from CD4+ and CD8+ T cells that recognize VOCs, including Omicron.

The substantial escape of Omicron variants 1 subvariants to neutralizing antibodies induced by vaccination and by previous infection (Cao et al., 2022) with greater risk of breakthrough infections, encouraged the production of bivalent COVID-19 vaccines based on the wild-type Spike protein combined with Omicron's Spike BA.1 or BA.4-5. Higher anti-Omicron BA. 1 titers were observed with vaccines containing Omicron Spike (mRNA-1273, Beta or Delta) comparing to the monovalent mRNA-1273 vaccine (Wuhan-1 SARS-COV-2 strain). However, titers against Omicron BA. 4/BA. 5 were lower than against BA.1 for all vaccine candidates (Branche et al., 2022). Another study that showed a bivalent mRNA-1273 vaccine comprising Omicron Spike BA.1 administered as a second booster induced higher antibody titers against Omicron BA.1 compared with the monovalent mRNA-127344 vaccine (Chalkias et al., 2022). In contrast, bivalent mRNA boosters that encode Omicron BA. 5 (Collier et al., 2023) or BA.4/BA.5 (Wang et al., 2022) and ancestral Spike showed similar anti-BA.5 or anti-BA. 4/BA. 5 nAb titers after boosting to the original monovalent and bivalent mRNA.

Thus, although the ability of bivalent vaccines to induce nAbs specific to VOCs and enhance protective immunity to SARS-COV-2 variants is still under investigation, it is possible that their ability to induce immune-mediated resistance to Omicron subvariants remains largely dependent on effector T cells.

In conclusion, the rapid evolution and prevalence of the Omicron variant, followed by its subvariants, with greater potential for immune escape, have posed the challenge of updating current vaccines with the S protein of emerging VOCs. However, vaccines employing ancestral SARS-COV-2 targets are still important for COVID-19 protection, until we have more conclusive results on vaccines adapted to the variants. Plasmid DNAS are low-cost vaccines that are easy to produce on an industrial scale. Furthermore, they are highly stable, facilitating their distribution in countries with limited infrastructure for distributing refrigerated and frozen vaccines. In addition, rapid adaptation to emerging/reemerging pathogens suggests that plasmid DNAs are a promising platform to be used as a booster in transmission waves with new COVID-19 VOCs.

The invention therefore provides the following aspects/embodiments:

1. Recombinant plasmid comprising the nucleic acid sequence as set forth in SEQ ID NO. 1 and the gene sequence encoding the antigen.

2. Recombinant plasmid, according to aspect 1, wherein the antigen is an antigenic protein of SARS-COV-2 or influenza viruses.

3. Recombinant plasmid, according to any of the aspects 1 to 2, wherein the antigen is selected from the group consisting of the antigenic protein of seasonal influenza H1 or H3 variant, spike protein of the SARS-COV-2 virus and NP protein of the influenza virus.

4. Recombinant plasmid, according to any of the aspects 1 to 3, wherein it comprises the nucleic acid sequence as set forth in SEQ ID NO. 3, 4 or 7.

5. Recombinant bivalent plasmid comprising the nucleic acid sequence as set forth in SEQ ID NO. 2 and the gene sequences encoding the antigens.

6. Recombinant bivalent plasmid, according to aspect 5, wherein it has the size of approximately 3.4 kb.

7. Recombinant bivalent plasmid, according to aspect 5, wherein such plasmid allows the insertion of two genes using two independent promoters.

8. Recombinant bivalent plasmid, according to any of the aspects 5 to 7, wherein the independent promoters are selected from the group consisting of simian vacuolating virus 40 promoter (SV40) and human cytomegalovirus promoter (CMV).

9. Recombinant bivalent plasmid, according to any of the aspects 5 to 8, wherein the antigen is selected from the group consisting of an antigenic protein of SARS-COV-2 virus, Influenza virus or a combination thereof.

10. Recombinant bivalent plasmid, according to any of the aspects 5 to 9, wherein the antigen is selected from the group consisting of the antigenic protein of seasonal influenza H1 or H3 variant, spike protein of the SARS-COV-2 virus and NP protein of the influenza virus.

11. Recombinant bivalent plasmid, according to any of the aspects 5 to 10, wherein it comprises the nucleic acid sequence as set forth in SEQ ID NO. 5.

12. Immunogenic composition comprising an immunologically acceptable amount of the plasmid as defined in any of the aspects 1 to 4 and a vehicle, excipient, pharmaceutically acceptable adjuvant.

13. Bivalent immunogenic composition comprising an immunologically acceptable amount of the plasmid as defined in any of the aspects 5 to 10 and a vehicle, excipient, pharmaceutically acceptable adjuvant.

14. Use of the plasmid as defined in any of the aspects 1 to 4 or of the immunogenic composition as defined by aspect 12, wherein it is for the production of a vaccine for prevention of high viral loads and moderate and severe clinical forms of the disease caused by the influenza virus or SARS-COV-2 viruses.

15. Use of the plasmid as defined in any of the aspects 5 to 11 or of the immunogenic composition as defined by aspect 13 wherein it is for the production of a vaccine for simultaneous prevention of high viral loads and moderate and severe clinical forms of the disease caused by SARS-COV-2 and Influenza viruses.

16. Kit comprising a first recipient comprising the plasmid as defined in any of the aspects 1 to 11 or a composition as defined in any of the aspects 12 to 13, wherein it is prepared to be administered in a prime dose and a second recipient comprising the plasmid as defined in any of the aspects 1 to 11 or the composition as defined in any of the aspects 12 to 13 wherein it is prepared to be administered as a booster dose, and instructions for use.

Thus, although only some embodiments/aspects of the present invention have been shown, it will be understood that various omissions, replacements, and changes to the vaccine platform can be made by a person skilled in the art, without departing from the scope of the present invention.

It is expressly anticipated that all combinations of the elements that perform the same function in substantially the same way to achieve the same results are within the scope of the invention. The replacement of elements from one described embodiment to another are also fully intended and contemplated.

It should also be understood that the drawings are not necessarily to scale but are conceptual in nature only. The intention is therefore to be limited, as indicated by the scope of the appended claims.

### REFERENCE

Wolff, J. A., Malone, R. W., Williams, P., Chong, W., Acsadi, G., Jani, A., & Felgner, P. L. (1990). Direct gene transfer into mouse muscle in vivo. Science, 247 (4949), 1465-1468.

We claim:

1. Recombinant plasmid characterized by the fact that it comprises the nucleic acid sequence as set forth in SEQ ID NO: 1 and the gene sequence encoding the antigen.

2. Recombinant plasmid, according to claim 1, characterized by the fact that the antigen is an antigenic protein of a SARS-CoV-2 or Influenza viruses.

3. Recombinant plasmid, according to claim 1, characterized by the fact that the antigen is selected from the group consisting of antigenic protein of the seasonal influenza H1 variant, spike protein of the SARS-COV-2 virus and NP protein of the influenza virus.

4. Recombinant plasmid, according to claim 1, characterized by the fact that it comprises the nucleic acid sequence as set forth in SEQ ID NOS: 3, 4 or 7.

5. Recombinant bivalent plasmid characterized by the fact that it comprises the nucleic acid sequence as set forth in SEQ ID NO: 2 and the gene sequences encoding the antigens.

6. Recombinant bivalent plasmid, according to claim 5, characterized by the fact that it has about 2,000 pb less than the commercial plasmid, presenting ˜3.4 kb.

7. Recombinant bivalent plasmid, according to claim 5, characterized by the fact that such plasmid allows the insertion of two genes using two independent promoters.

8. Recombinant bivalent plasmid, according to claim 5, characterized by the fact that the independent promoters are selected from the group consisting of the simian vacuolating virus 40 promoter (SV40) and human cytomegalovirus promoter (CMV).

9. Recombinant bivalent plasmid, according to claim 5, characterized by the fact that the antigen is selected from the group consisting of an antigenic protein of the SARS-COV-2 virus, Influenza virus or a combination thereof.

10. Recombinant bivalent plasmid, according to claim 5, characterized by the fact that the antigen is selected from the group consisting of the antigenic protein of the seasonal influenza H1 variant, spike protein of the SARS-COV-2 virus and NP protein of the influenza virus.

11. Recombinant bivalent plasmid, according to claim 5, characterized by the fact that it comprises the nucleic acid sequence as set forth in SEQ ID NO. 5.

12. Immunogenic composition characterized by the fact that it comprises an immunologically acceptable amount of the plasmid as defined in claim 1 and a vehicle, excipient, pharmaceutically acceptable adjuvant.

13. Bivalent immunogenic composition characterized by the fact that it comprises an immunologically acceptable amount of the plasmid as defined in claim 5 and a vehicle, excipient, pharmaceutically acceptable adjuvant.

14. A method of using the plasmid as defined claim 1 characterized by the fact that it is for the production of a vaccine to prevent high viral loads and moderate and severe clinical forms of the disease caused by the influenza virus or SARS-COV-2 viruses.

15. A method of using the plasmid as defined in claim 5 characterized by the fact that it is for the production of a vaccine for simultaneous prevention of high viral loads and moderate and severe clinical forms of the disease caused by SARS-COV-2 and Influenza viruses.

16. A kit characterized by the fact that it comprises a first recipient comprising the plasmid as defined in claim 1 or a composition comprising the plasmid prepared to be administered in a prime dose and a second recipient comprising the plasmid or a composition comprising the plasmid prepared to be administered as a booster dose, and instructions for use.

17. A method of preventing high viral loads and moderate and severe clinical forms of disease caused by the influenza virus or SARS-COV-2 viruses comprising administering the bivalent immunogenic composition of claim 12 to a subject in need thereof.

18. A method of preventing high viral loads and moderate and severe clinical forms of disease caused by the influenza virus or SARS-COV-2 viruses comprising administering the bivalent immunogenic composition of claim 13 to a subject in need thereof.

19. A kit characterized by the fact that it comprises a first recipient comprising the plasmid as defined in claim 5 or a composition comprising the plasmid prepared to be administered in a prime dose and a second recipient comprising the plasmid or a composition comprising the plasmid prepared to be administered as a booster dose, and instructions for use.


SEQUENCE LISTING (38 sequences)

SEQ ID NO: 1 - DNA, 3014 bp, synthetic construct
  source 1..3014: mol_type = other DNA
  misc_feature 1..3014: note = Plasmdeo base (pCTV1)
ttaatacgactcactatagggagacccaagctggctagcgtttaaacttaagcttggtaccgagctcggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcccgggtttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaa

SEQ ID NO: 2 - DNA, 3456 bp, synthetic construct
  source 1..3456: mol_type = other DNA
  misc_feature 1..3456: note = Plasmdeo para vacina bivalente - dois promotores (pCTV2)
gatctgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaagaattctgcagatatccagcacagtggcggccgctcgagtctagaaacttgtttattgcagcttataatggttacaaataaagcaatagcatcacaaatttcacaaataaagcatttttttcactgcattctagttgtggtttgtccaaactcatcaatgtatcttagtcgacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaacccgggccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgggtttaaacgggccaccacactggactagtggatccgagctcggtaccaagcttaagtttaaacgctagccagcttgggtctccctatagtgagtcgtattaatttcgataagccagtaagcagtgggttctctagttagccagagagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaacgcgtatatctggcccgtacatcgcgaagcagcgcaaaacgcctaaccctaagcagattcttcatgcaattgtcggtcaagccttgccttgttgtagcttaaattttgctcgcgcactactcagcgacctccaacacacaagcagggagcagatactggcttaactatgcggcatcagagcagattgtactgagagtgcaccataggggatcggga

SEQ ID NO: 3 - DNA, 4548 bp, synthetic construct
  source 1..4548: mol_type = other DNA
  misc_feature 1..4548: note = pCTV1-NP / pCTV1-NP
ttaatacgactcactatagggagacccaagctggctagcgtttaaacttaagcttggtaccgagctcggatccactagtccagtgtggtggaattctgcagatatccagcacagtggcggccgcatggcgtctcaaggcaccaaacgatcttacgaacagatggagactgatggagaacgccagaatgccactgaaatcagagcatccgtcggaaaaatgattggtggaattggacgattctacatccaaatgtgcaccgaactcaaactcagtgattatgagggacggttgatccaaaacagcttaacaatagagagaatggtgctctctgcttttgacgaaaggagaaataaataccttgaagaacatcccagtgcggggaaagatcctaagaaaactggaggacctatatacaggagagtaaacggaaagtggatgagagaactcatcctttatgacaaagaagaaataaggcgaatctggcgccaagctaataatggtgacgatgcaacggctggtctgactcacatgatgatctggcattccaatttgaatgatgcaacttatcagaggacaagagctcttgttcgcaccggaatggatcccaggatgtgctctctgatgcaaggttcaactctccctaggaggtctggagccgcaggtgctgcagtcaaaggagttggaacaatggtgatggaattggtcagaatgatcaaacgtgggatcaatgatcggaacttctggaggggtgagaatggacgaaaaacaagaattgcttatgaaagaatgtgcaacattctcaaagggaaatttcaaactgctgcacaaaaagcaatgatggatcaagtgagagagagccggaacccagggaatgctgagttcgaagatctcacttttctagcacggtctgcactcatattgagagggtcggttgctcacaagtcctgcctgcctgcctgtgtgtatggacctgccgtagccagtgggtacgactttgaaagggagggatactctctagtcggaatagaccctttcagactgcttcaaaacagccaagtgtacagcctaatcagaccaaatgagaatccagcacacaagagtcaactggtgtggatggcatgccattctgccgcatttgaagatctaagagtattaagcttcatcaaagggacgaaggtgctcccaagagggaagctttccactagaggagttcaaattgcttccaatgaaaatatggagactatggaatcaagtacacttgaactgagaagcaggtactgggccataaggaccagaagtggaggaaacaccaatcaacagagggcatctgcgggccaaatcagcatacaacctacgttctcagtacagagaaatctcccttttgacagaacaaccattatggcagcattcaatgggaatacagaggggagaacatctgacatgaggaccgaaatcataaggatgatggaaagtgcaagaccagaagatgtgtctttccaggggcggggagtcttcgagctctcggacgaaaaggcagcgagcccgatcgtgccttcctttgacatgagtaatgaaggatcttatttcttcggagacaatgcagaggagtacgacaattaatctagagtggtggtggtgatggtgatgatgatgatgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcccgggtttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaa

SEQ ID NO: 4 - DNA, 6793 bp, synthetic construct
  source 1..6793: mol_type = other DNA
  misc_feature 1..6793: note = pCTV1_HsSpike
ttaatacgactcactatagggagacccaagctggctagcaaataagagagaaaagaagagtaagaagaaatataagagccacctctagaatgttcgtgttcttagtcctcctccctctggtgagctcgcagtgcgtgaatctgaccacccggacccagctgcctcctgcgtacaccaacagcttcacccgcggagtttattaccctgacaaggtattccggagcagcgtgctgcacagcacacaggatctcttcctgccattcttcagcaacgtgacatggtttcacgctatccacgtgtcaggcacaaatggaaccaagagattcgacaaccccgtgctgcctttcaacgacggcgtttacttcgcgagcaccgagaaatcgaatatcatcagaggatggatcttcggcaccacactggacagcaagacccagagcctgcttatcgtaaacaatgccacaaacgtggtgatcaaggtgtgcgagttccagttttgtaacgaccccttcctgggagtttactaccacaagaacaacaaaagctggatggaaagcgagttcagagtctacagctctgccaacaattgcactttcgagtacgtgtcccagcccttcctcatggacctggaaggaaagcagggcaacttcaagaacctgagagaattcgtgttcaagaacatcgatggatacttcaagatttactccaagcacacccctatcaacctggtgagagatctgccccagggctttagcgccctggaacctctggtggacctgccgatcggaatcaacatcacgcggttccagaccctgctggccctccatagatcttatctgaccccaggcgacagcagctccggatggacagccggcgccgccgcctactacgtgggatatctgcagcccaggaccttcctgctgaagtacaacgagaatggtacaatcacagacgccgtggattgcgccctggaccccctgagcgagacaaaatgcacactgaagagcttcactgttgaaaagggcatataccagaccagcaacttcagagtccagcctaccgagagcatcgtgcggtttcccaacatcactaacctgtgtcctttcggcgaggtctttaacgccacaagattcgcctctgtgtacgcttggaacagaaagagaatcagcaactgcgtggccgattacagcgtgctgtacaacagcgcgagcttcagcacattcaaatgttacggcgtgagccctacaaagctgaatgacctgtgcttcacgaacgtgtacgccgatagcttcgtgatccggggcgacgaagtgcggcagatcgcccctgggcagaccggcaagatcgctgattacaactacaagctgccagacgacttcaccggctgtgtgatcgcctggaacagcaacaacctggacagcaaggtgggcggaaactacaactacctgtatcggctgtttcgaaagagcaacctgaagcctttcgagcgggatatcagcaccgagatctaccaggctggcagcacaccttgcaacggcgtggaaggattcaactgctacttccctctgcaatcttatggcttccagcctacaaatggcgtcggctaccagccttacagagtggtggtgctgagcttcgagctgcttcatgcccctgccaccgtctgcggacctaagaaaagcaccaatctggtgaaaaataagtgtgtcaacttcaactttaatggcctgaccggcaccggcgtcctgaccgagtctaacaaaaagttcctgccttttcaacagttcggcagagacatcgcagacacaacagatgccgtgagagaccctcagacacttgagatcctggacatcaccccttgttccttcggcggcgtatccgtgattacccctggcacaaataccagcaaccaggtggccgtgctgtaccaggacgtgaactgcaccgaagtgcctgttgccatccacgccgatcagctgacacctacatggcgggtgtacagcacaggcagcaacgtgttccagaccagagccggatgtctgatcggagccgagcacgtgaataactcctacgaatgtgacattccaatcggtgccggcatctgtgcctcttaccagacccagaccaacagccctgcctctgtcgcctctcagagcatcatcgcctatacaatgtccctgggcgccgagaacagcgtggcctattctaacaatagcatcgctatccctaccaacttcacgatctctgttaccacagagatcctgccagtgagcatgaccaaaaccagcgtggactgcaccatgtacatctgcggcgacagcacagaatgcagcaacctgctgctgcaatacggcagcttctgcacccagctgaacagagccctgaccggcatcgccgtggaacaggacaagaatacccaggaggtgttcgcccaggtgaagcaaatctacaagacccctccaattaaggacttcggcggcttcaacttcagccagatcctgcccgatccctccaagcccagcaagaggtctttcatagaggacctgctgttcaacaaggtgaccctcgccgatgccggctttatcaagcagtacggggactgcctgggcgacatcgcggctagagacctgatttgcgcccaaaagtttaacggcctgacagtgctgcctcctctgctgactgatgagatgatcgctcagtacaccagcgccctgctggctggcaccatcaccagtgggtggacctttggagccggagctgctctgcagatccccttcgccatgcagatggcctaccggttcaatggaattggcgttacacagaatgtgctctatgaaaaccagaagctgatcgctaaccagttcaacagtgccatcggcaagatccaggactctctgagctcaaccgccagcgccctgggcaagctgcaggatgtggtgaaccagaacgctcaagctctgaacaccctggtgaagcagctgtcttctaattttggcgccatttcatctgtcttgaacgacatcctgagcagactggacccccccgaggccgaagtgcagatcgacagactgatcaccggcagactgcagtctctgcagacatacgtgacccagcagctgatccgcgccgcggagatcagagccagcgccaacctggccgccaccaagatgagcgagtgcgtcctgggccagtccaaaagagtggatttctgcggcaagggctaccacctaatgtcctttccacagagcgcccctcacggcgtagtgttcctgcacgtgacttacgtgcccgcacaggagaagaactttaccacagcccctgctatttgtcacgacggcaaggcccacttccctagagagggcgtttttgtgagcaacggcacccactggtttgtgacccagcggaacttctacgagcctcagattatcaccaccgataacacattcgtgtccggcaactgcgatgtggtgatcggcatcgtgaacaacacagtgtacgaccctctgcaaccagaacttgatagcttcaaggaggagctggataagtactttaagaaccacacatctccagacgtggatctgggcgatatcagcggaatcaacgcctccgtggtgaacatccagaaggaaatcgaccggctgaatgaagtggccaaaaatctgaacgaaagtctgatcgacctgcaagagctgggtaaatacgaacagtacatcaaatggccttggtacatctggctgggcttcatcgccggactgatcgccattgtgatggtgaccatcatgctgtgctgcatgacctcctgctgctcttgtctgaaaggatgttgcagctgcggctcttgctgcaagtttgacgaggatgacagtgaacccgtgctgaagggcgtgaagctgcactacacatgagtcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcccgggtttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaa

SEQ ID NO: 5 - DNA, 8738 bp, synthetic construct
  source 1..8738: mol_type = other DNA
  misc_feature 1..8738: note = pCTV2_NP(CMV)_hsSpike(SV40)
gatctgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaagaattctgcagatatccagcacagtggcggccgcgcaaataagagagaaaagaagagtaagaagaaatataagagccacctctagaatgttcgtgttcttagtcctcctccctctggtgagctcgcagtgcgtgaatctgaccacccggacccagctgcctcctgcgtacaccaacagcttcacccgcggagtttattaccctgacaaggtattccggagcagcgtgctgcacagcacacaggatctcttcctgccattcttcagcaacgtgacatggtttcacgctatccacgtgtcaggcacaaatggaaccaagagattcgacaaccccgtgctgcctttcaacgacggcgtttacttcgcgagcaccgagaaatcgaatatcatcagaggatggatcttcggcaccacactggacagcaagacccagagcctgcttatcgtaaacaatgccacaaacgtggtgatcaaggtgtgcgagttccagttttgtaacgaccccttcctgggagtttactaccacaagaacaacaaaagctggatggaaagcgagttcagagtctacagctctgccaacaattgcactttcgagtacgtgtcccagcccttcctcatggacctggaaggaaagcagggcaacttcaagaacctgagagaattcgtgttcaagaacatcgatggatacttcaagatttactccaagcacacccctatcaacctggtgagagatctgccccagggctttagcgccctggaacctctggtggacctgccgatcggaatcaacatcacgcggttccagaccctgctggccctccatagatcttatctgaccccaggcgacagcagctccggatggacagccggcgccgccgcctactacgtgggatatctgcagcccaggaccttcctgctgaagtacaacgagaatggtacaatcacagacgccgtggattgcgccctggaccccctgagcgagacaaaatgcacactgaagagcttcactgttgaaaagggcatataccagaccagcaacttcagagtccagcctaccgagagcatcgtgcggtttcccaacatcactaacctgtgtcctttcggcgaggtctttaacgccacaagattcgcctctgtgtacgcttggaacagaaagagaatcagcaactgcgtggccgattacagcgtgctgtacaacagcgcgagcttcagcacattcaaatgttacggcgtgagccctacaaagctgaatgacctgtgcttcacgaacgtgtacgccgatagcttcgtgatccggggcgacgaagtgcggcagatcgcccctgggcagaccggcaagatcgctgattacaactacaagctgccagacgacttcaccggctgtgtgatcgcctggaacagcaacaacctggacagcaaggtgggcggaaactacaactacctgtatcggctgtttcgaaagagcaacctgaagcctttcgagcgggatatcagcaccgagatctaccaggctggcagcacaccttgcaacggcgtggaaggattcaactgctacttccctctgcaatcttatggcttccagcctacaaatggcgtcggctaccagccttacagagtggtggtgctgagcttcgagctgcttcatgcccctgccaccgtctgcggacctaagaaaagcaccaatctggtgaaaaataagtgtgtcaacttcaactttaatggcctgaccggcaccggcgtcctgaccgagtctaacaaaaagttcctgccttttcaacagttcggcagagacatcgcagacacaacagatgccgtgagagaccctcagacacttgagatcctggacatcaccccttgttccttcggcggcgtatccgtgattacccctggcacaaataccagcaaccaggtggccgtgctgtaccaggacgtgaactgcaccgaagtgcctgttgccatccacgccgatcagctgacacctacatggcgggtgtacagcacaggcagcaacgtgttccagaccagagccggatgtctgatcggagccgagcacgtgaataactcctacgaatgtgacattccaatcggtgccggcatctgtgcctcttaccagacccagaccaacagccctgcctctgtcgcctctcagagcatcatcgcctatacaatgtccctgggcgccgagaacagcgtggcctattctaacaatagcatcgctatccctaccaacttcacgatctctgttaccacagagatcctgccagtgagcatgaccaaaaccagcgtggactgcaccatgtacatctgcggcgacagcacagaatgcagcaacctgctgctgcaatacggcagcttctgcacccagctgaacagagccctgaccggcatcgccgtggaacaggacaagaatacccaggaggtgttcgcccaggtgaagcaaatctacaagacccctccaattaaggacttcggcggcttcaacttcagccagatcctgcccgatccctccaagcccagcaagaggtctttcatagaggacctgctgttcaacaaggtgaccctcgccgatgccggctttatcaagcagtacggggactgcctgggcgacatcgcggctagagacctgatttgcgcccaaaagtttaacggcctgacagtgctgcctcctctgctgactgatgagatgatcgctcagtacaccagcgccctgctggctggcaccatcaccagtgggtggacctttggagccggagctgctctgcagatccccttcgccatgcagatggcctaccggttcaatggaattggcgttacacagaatgtgctctatgaaaaccagaagctgatcgctaaccagttcaacagtgccatcggcaagatccaggactctctgagctcaaccgccagcgccctgggcaagctgcaggatgtggtgaaccagaacgctcaagctctgaacaccctggtgaagcagctgtcttctaattttggcgccatttcatctgtcttgaacgacatcctgagcagactggacccccccgaggccgaagtgcagatcgacagactgatcaccggcagactgcagtctctgcagacatacgtgacccagcagctgatccgcgccgcggagatcagagccagcgccaacctggccgccaccaagatgagcgagtgcgtcctgggccagtccaaaagagtggatttctgcggcaagggctaccacctaatgtcctttccacagagcgcccctcacggcgtagtgttcctgcacgtgacttacgtgcccgcacaggagaagaactttaccacagcccctgctatttgtcacgacggcaaggcccacttccctagagagggcgtttttgtgagcaacggcacccactggtttgtgacccagcggaacttctacgagcctcagattatcaccaccgataacacattcgtgtccggcaactgcgatgtggtgatcggcatcgtgaacaacacagtgtacgaccctctgcaaccagaacttgatagcttcaaggaggagctggataagtactttaagaaccacacatctccagacgtggatctgggcgatatcagcggaatcaacgcctccgtggtgaacatccagaaggaaatcgaccggctgaatgaagtggccaaaaatctgaacgaaagtctgatcgacctgcaagagctgggtaaatacgaacagtacatcaaatggccttggtacatctggctgggcttcatcgccggactgatcgccattgtgatggtgaccatcatgctgtgctgcatgacctcctgctgctcttgtctgaaaggatgttgcagctgcggctcttgctgcaagtttgacgaggatgacagtgaacccgtgctgaagggcgtgaagctgcactacacatgactcgagtctagaaacttgtttattgcagcttataatggttacaaataaagcaatagcatcacaaatttcacaaataaagcatttttttcactgcattctagttgtggtttgtccaaactcatcaatgtatcttagtcgacgtcaggtggcacttttcggggaaatgtgcgcggaacccctatttgtttatttttctaaatacattcaaatatgtatccgctcatgagacaataaccctgataaatgcttcaataatattgaaaaaggaagagtatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaacccgggccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgggtttaaacgggcccaccacactggactagtggatccgagctcggtaccttaattgtcgtactcctctgcattgtctccgaagaaataagatccttcattactcatgtcaaaggaaggcacgatcgggctcgctgccttttcgtccgagagctcgaagactccccgcccctggaaagacacatcttctggtcttgcactttccatcatccttatgatttcggtcctcatgtcagatgttctcccctctgtattcccattgaatgctgccataatggttgttctgtcaaaagggagatttctctgtactgagaacgtaggttgtatgctgatttggcccgcagatgccctctgttgattggtgtttcctccacttctggtccttatggcccagtacctgcttctcagttcaagtgtacttgattccatagtctccatattttcattggaagcaatttgaactcctctagtggaaagcttccctcttgggagcaccttcgtccctttgatgaagcttaatactcttagatcttcaaatgcggcagaatggcatgccatccacaccagttgactcttgtgtgctggattctcatttggtctgattaggctgtacacttggctgttttgaagcagtctgaaagggtctattccgactagagagtatccctccctttcaaagtcgtacccactggctacggcaggtccatacacacaggcaggcaggcaggacttgtgagcaaccgaccctctcaatatgagtgcagaccgtgctagaaaagtgagatcttcgaactcagcattccctgggttccggctctctctcacttgatccatcattgctttttgtgcagcagtttgaaatttccctttgagaatgttgcacattctttcataagcaattcttgtttttcgtccattctcacccctccagaagttccgatcattgatcccacgtttgatcattctgaccaattccatcaccattgttccaactcctttgactgcagcacctgcggctccagacctcctagggagagttgaaccttgcatcagagagcacatcctgggatccattccggtgcgaacaagagctcttgtcctctgataagttgcatcattcaaattggaatgccagatcatcatgtgagtcagaccagccgttgcatcgtcaccattattagcttggcgccagattcgccttatttcttctttgtcataaaggatgagttctctcatccactttccgtttactctcctgtatataggtcctccagttttcttaggatctttccccgcactgggatgttcttcaaggtatttatttctcctttcgtcaaaagcagagagcaccattctctctattgttaagctgttttggatcaaccgtccctcataatcactgagtttgagttcggtgcacatttggatgtagaatcgtccaattccaccaatcatttttccgacggatgctctgatttcagtggcattctggcgttctccatcagtctccatctgttcgtaagatcgtttggtgccttgagacgccatgctagccagcttgggtctccctatagtgagtcgtattaatttcgataagccagtaagcagtgggttctctagttagccagagagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaacgcgtatatctggcccgtacatcgcgaagcagcgcaaaacgcctaaccctaagcagattcttcatgcaattgtcggtcaagccttgccttgttgtagcttaaattttgctcgcgcactactcagcgacctccaacacacaagcagggagcagatactggcttaactatgcggcatcagagcagattgtactgagagtgcaccataggggatcggga

SEQ ID NO: 6 - DNA, 4770 bp, synthetic construct
  source 1..4770: mol_type = other DNA
  misc_feature 1..4770: note = pCTV2-GFP-RFP
tcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcatgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttcggttatggtgttcaatgctttgcgagatacccagatcatatgaaacagcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaataaggtaccgagctcggatccactagtccagtgtggtgggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcccgggtttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagtcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatactcttcctttttcaatattattgaagcatttatcagggttattgtctcatgagcggatacatatttgaatgtatttagaaaaataaacaaataggggttccgcgcacatttccccgaaaagtgccacctgacgtcgactaagatacattgatgagtttggacaaaccacaactagaatgcagtgaaaaaaatgctttatttgtgaaatttgtgatgctattgctttatttgtaaccattataagctgcaataaacaagtttctagattaggcgccggtggagtggcggccctcggcgcgctcgtactgttccacgatggtgtagtcctcgttgtgggaggtgatgtccagcttgatgtcggtcttgtaggcgccgggcagctgcacgggcttcttggccatgtaggtggtcttgacctcggcgtcgtagtggccgccgtccttcagcttcagcctcatcttgatctcgcccttcagggcgccgtcctcggggtacatccgctcggtggaggcctcccagcccatggtcttcttctgcattacggggccgtcggaggggaagttggtgccgcgcagcttcaccttgtagatgaactcgccgtcctgcagggaggagtcctgggtcacggtcaccacgccgccgtcctcgaagttcatcacgcgctcccacttgaagccctcggggaaggacagcttcaagtagtcggggatgtcggcggggtgcttcacgtaggccttggagccgtactggaactgaggggacaggatgtcccaggcgaagggcagggggccgcccttggtcaccttcagcttggcggtctgggtgccctcgtaggggcggccctcgccctcgccctcgatctcgaactcgtggccgttcacggagccctccatgcgcaccttgaagcgcatgaactccttgatgacgtcctcggaggaggccatctcgagcggccgccactgtgctggatatctgcagaattctttgcaaaagcctaggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacagatc

SEQ ID NO: 7 - DNA, 4707 bp, synthetic construct
  source 1..4707: mol_type = other DNA
  misc_feature 1..4707: note = pCTV1-H1
ttaatacgactcactatagggagacccaagctggctagcgtttaaacttaagcttggtaccgagctcggatccactagtccagtgtggtggaattctgcagatatccagcacagtggcggccgcatgaaggcaaacctactggtcctgttatgtgcacttgcagctgcagatgcagacacaatatgtataggctaccatgcgaacaattcaaccgacactgttgacacagtactcgagaagaatgtgacagtgacacactctgttaacctgctcgaagacagccacaacggaaaactatgtagattaaaaggaatagccccactacaattggggaaatgtaacatcgccggatggctcttgggaaacccagaatgcgacccactgcttccagtgagatcatggtcctacattgtagaaacaccaaactctgagaatggaatatgttatccaggagatttcatcgactatgaggagctgagggagcaattgagctcagtgtcatcattcgaaagattcgaaatatttcccaaagaaagctcatggcccaaccacaacacaaacggagtaacggcagcatgctcccatgaggggaaaagcagtttttacagaaatttgctatggctgacggagaaggagggctcatacccaaagctgaaaaattcttatgtgaacaaaaaagggaaagaagtccttgtactgtggggtattcatcacccgcctaacagtaaggaacaacagaatctctatcagaatgaaaatgcttatgtctctgtagtgacttcaaattataacaggagatttaccccggaaatagcagaaagacccaaagtaagagatcaagctgggaggatgaactattactggaccttgctaaaacccggagacacaataatatttgaggcaaatggaaatctaatagcaccaatgtatgctttcgcactgagtagaggctttgggtccggcatcatcacctcaaacgcatcaatgcatgagtgtaacacgaagtgtcaaacacccctgggagctataaacagcagtctcccttaccagaatatacacccagtcacaataggagagtgcccaaaatacgtcaggagtgccaaattgaggatggttacaggactaaggaacaatccgtccattcaatccagaggtctatttggagccattgccggttttattgaagggggatggactggaatgatagatggatggtatggttatcatcatcagaatgaacagggatcaggctatgcagcggatcaaaaaagcacacaaaatgccattaacgggattacaaacaaggtgaacactgttatcgagaaaatgaacattcaattcacagctgtgggtaaagaattcaacaaattagaaaaaaggatggaaaatttaaataaaaaagttgatgatggatttctggacatttggacatataatgcagaattgttagttctactggaaaatgaaaggactctggatttccatgactcaaatgtgaagaatctgtatgagaaagtaaaaagccaattaaagaataatgccaaagaaatcggaaatggatgttttgagttctaccacaagtgtgacaatgaatgcatggaaagtgtaagaaatgggacttatgattatcccaaatattcagaagagtcaaagttgaacagggaaaaggtagatggagtgaaattggaatcaatggggatctatcagattctggcgatctactcaactgtcgccagttcactggtgcttttggtctccctgggggcaatcagtttctggatgtgttctaatggatctttgcagtgcagaatatgcatctgatctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcccgggtttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaa

SEQ ID NO: 8 - DNA, 19 bp, synthetic construct
  source 1..19: mol_type = other DNA
  misc_feature 1..19: note = Iniciador 5' do gene B-actin - forward
cgatgccctgaggctcttt

SEQ ID NO: 9 - DNA, 20 bp, synthetic construct
  source 1..20: mol_type = other DNA
  misc_feature 1..20: note = Iniciador 3' do gene B-actin - reverse
tggatgccacaggattccat

SEQ ID NO: 10 - DNA, 19 bp, synthetic construct
  source 1..19: mol_type = other DNA
  misc_feature 1..19: note = Iniciador 5' do gene CCL2 - forward
tggctcagccagatgcagt

SEQ ID NO: 11 - DNA, 20 bp, synthetic construct
  source 1..20: mol_type = other DNA
  misc_feature 1..20: note = Iniciador 3' do gene CCL2 - reverse
ttgggatcatcttgctggtg

SEQ ID NO: 12 - DNA, 22 bp, synthetic construct
  source 1..22: mol_type = other DNA
  misc_feature 1..22: note = Iniciador 5' do gene CCL5 - forward
caagtgctccaatcttgcagtc

SEQ ID NO: 13 - DNA, 20 bp, synthetic construct
  source 1..20: mol_type = other DNA
  misc_feature 1..20: note = Iniciador 3' do gene CCL5 - reverse
ttctctgggttggcacacac

SEQ ID NO: 14 - DNA, 19 bp, synthetic construct
  source 1..19: mol_type = other DNA
  misc_feature 1..19: note = Iniciador 5' do gene CXCL9 - forward
aatgcacgatgctcctgca

SEQ ID NO: 15 - DNA, 24 bp, synthetic construct
  source 1..24: mol_type = other DNA
  misc_feature 1..24: note = Iniciador 3' do gene CXCL9 - reverse
aggtctttgagggatttgtagtgg

SEQ ID NO: 16 - DNA, 19 bp, synthetic construct
  source 1..19: mol_type = other DNA
  misc_feature 1..19: note = Iniciador 5' do gene CXCL10 - forward
gccgtcattttctgcctca

SEQ ID NO: 17 - DNA, 19 bp, synthetic construct
  source 1..19: mol_type = other DNA
  misc_feature 1..19: note = Inciador 3' do gene CXCL10 - reverse
cgtccttgcgagagggatc

SEQ ID NO: 18 - DNA, 20 bp, synthetic construct
  source 1..20: mol_type = other DNA
  misc_feature 1..20: note = Iniciador 5' do gene GAPDH - forward
ggcaaattcaacggcacagt

SEQ ID NO: 19 - DNA, 19 bp, synthetic construct
  source 1..19: mol_type = other DNA
  misc_feature 1..19: note = Iniciador 3' do gene GAPDH - reverse
agatggtgatgggcttccc

SEQ ID NO: 20 - DNA, 20 bp, synthetic construct
  source 1..20: mol_type = other DNA
  misc_feature 1..20: note = Iniciador 5' do gene HPRT - forward
ggcaaattcaacggcacagt

SEQ ID NO: 21 - DNA, 19 bp, synthetic construct
  source 1..19: mol_type = other DNA
  misc_feature 1..19: note = Iniciador 3' do gene HPRT - reverse
agatggtgatgggcttccc

SEQ ID NO: 22 - DNA, 21 bp, synthetic construct
  source 1..21: mol_type = other DNA
  misc_feature 1..21: note = Iniciador 5' do gene IFN-B - forward
cagctccaagaaaggacgaac

SEQ ID NO: 23 - DNA, 21 bp, synthetic construct
  source 1..21: mol_type = other DNA
  misc_feature 1..21: note = Iniciador 3' do gene IFN-B - reverse
ggcagtgtaactcttctgcat

SEQ ID NO: 24 - DNA, 22 bp, synthetic construct
  source 1..22: mol_type = other DNA
  misc_feature 1..22: note = Iniciador 5' do gene IFN-Y - forward
aacgctacacactgcatcttgg

SEQ ID NO: 25 - DNA, 18 bp, synthetic construct
  source 1..18: mol_type = other DNA
  misc_feature 1..18: note = Iniciador 3' do gene IFN-Y - reverse
gccgtggcagtaacagcc

SEQ ID NO: 26 - DNA, 24 bp, synthetic construct
  source 1..24: mol_type = other DNA
  misc_feature 1..24: note = Iniciador 5' do gene IL-1B - forward
acctgtcctgtgtaatgaaagacg

SEQ ID NO: 27 - DNA, 20 bp, synthetic construct
  source 1..20: mol_type = other DNA
  misc_feature 1..20: note = Iniciador 3' do gene IL-1B - reverse
tgggtattgcttgggatcca

SEQ ID NO: 28 - DNA, 22 bp, synthetic construct
  source 1..22: mol_type = other DNA
  misc_feature 1..22: note = Iniciador 5' do gene IL-5 - forward
aaagagaagtgtggcgaggaga

SEQ ID NO: 29 - DNA, 23 bp, synthetic construct
  source 1..23: mol_type = other DNA
  misc_feature 1..23: note = Iniciador 3' do gene IL-5 - reverse
caccaaggaactcttgcaggtaa

SEQ ID NO: 30 - DNA, 21 bp, synthetic construct
  source 1..21: mol_type = other DNA
  misc_feature 1..21: note = Iniciador 5' do gene IL-6 - forward
tgttctctgggaaatcgtgga

SEQ ID NO: 31 - DNA, 24 bp, synthetic construct
  source 1..24: mol_type = other DNA
  misc_feature 1..24: note = Iniciador 3' do gene IL-6 - reverse
aagtgcatcatcgttgttcataca

SEQ ID NO: 32 - DNA, 21 bp, synthetic construct
  source 1..21: mol_type = other DNA
  misc_feature 1..21: note = Iniciador 5' do gene IL-12 p40 - forward
tggtttgccatcgttttgctg

SEQ ID NO: 33 - DNA, 22 bp, synthetic construct
  source 1..22: mol_type = other DNA
  misc_feature 1..22: note = Iniciador 3' do gene IL-12 p40 - reverse
acaggtgaggttcactgtttct

SEQ ID NO: 34 - DNA, 23 bp, synthetic construct
  source 1..23: mol_type = other DNA
  misc_feature 1..23: note = Iniciador 5' do gene TNF-a - forward
ccctcacactcagatcatcttct

SEQ ID NO: 35 - DNA, 19 bp, synthetic construct
  source 1..19: mol_type = other DNA
  misc_feature 1..19: note = Iniciador 3' do gene TNF-a - reverse
gctacgacgtgggctacag

SEQ ID NO: 36 - DNA, 26 bp, synthetic construct
  source 1..26: mol_type = other DNA
  misc_feature 1..26: note = Iniciador 5' do gene E gene - forward
acaggtacgttaatagttaatagcgt

SEQ ID NO: 37 - DNA, 22 bp, synthetic construct
  source 1..22: mol_type = other DNA
  misc_feature 1..22: note = Iniciador 3' do gene E gene - reverse
atattgcagcagtacgcacaca

SEQ ID NO: 38 - DNA, 26 bp, synthetic construct
  misc_feature 1..26: note = Sonda do E gene (marcador FAM na extremidade 5' e marcador BBQ naextremidade 3')
  source 1..26: mol_type = other DNA
acactagccatccttactgcgcttcg